(19) United States
(12) Patent Application Publication
(10) Pub. No.: US 2023/0296635 A1
(43) Pub. Date: Sep. 21, 2023

(54) VIRION OSCILLATOR MICROARRAYS, METHODS, AND RELATED ASPECTS FOR DETERMINING BINDING KINETICS OF LIGANDS

(71) Applicant: ARIZONA BOARD OF REGENTS ON BEHALF OF ARIZONA STATE UNIVERSITY, Scottsdale, AZ (US)

(72) Inventors: Shaopeng WANG, Chandler, AZ (US); Zijian WAN, Tempe, AZ (US); Heng ZHU, Baltimore, MD (US)

(73) Assignee: ARIZONA BOARD OF REGENTS ON BEHALF OF ARIZONA STATE UNIVERSITY, Scottsdale, AZ (US)

(21) Appl. No.: 18/185,808

(22) Filed: Mar. 17, 2023

Related U.S. Application Data
(60) Provisional application No. 63/321,465, filed on Mar. 18, 2022.

(51) Int. Cl. G01N 33/94; G01N 33/543; G01N 21/64

(52) U.S. Cl. CPC G01N 33/94; G01N 33/54373; G01N 21/6428; G01N 2333/035; G01N 2500/04; G01N 2333/726; G01N 2021/6439

(57) ABSTRACT

Provided herein are methods of determining binding kinetics of a ligand. In some embodiments, the methods include contacting the ligand with a first surface of a substrate, which first surface comprises an electrically conductive coating and a population of virions connected to the first surface via one or more linker moieties, wherein viral envelopes of the virions display one or more proteins that bind, or are capable of binding, to the ligand, applying an alternating current electric field to the substrate to induce the virions to oscillate proximal to the first surface of the substrate, and detecting changes in oscillation amplitudes of the virions over a duration. Related virion oscillator array devices, systems and computer readable media are also provided.

### CROSS-REFERENCE TO RELATED APPLICATIONS

This application claims the benefit of U.S. Provisional Application No. 63/321,465 filed Mar. 18, 2022, the disclosure of which is incorporated herein in its entirety.

### STATEMENT OF GOVERNMENT SUPPORT

This invention was made with government support under R33 CA235294 awarded by the National Institutes of Health. The government has certain rights in the invention.

### BACKGROUND

Transmembrane proteins, such as G-protein-coupled receptors (GPCRs), are important for many cellular functions. They are also popular drug targets for various diseases, including cancer. For both understanding cellular functions and drug development, it is often necessary to measure their binding activities with molecular ligands and drug candidates. However, this has been a difficult task because of two challenges. First, transmembrane proteins are difficult to extract and purify, and they often lose their native conformations after isolation from the cellular membranes. Second, even if a membrane protein is successfully isolated, it remains challenging to measure its binding to ligands, especially with small molecule ligands. Small molecules comprise approximately 90% of the current drugs, but their binding kinetics cannot be easily measured with the existing detection technologies.

Accordingly, there is a need for effective techniques for measuring binding activities of molecular ligands, including drug candidates, with membrane proteins.

### SUMMARY

This disclosure describes virion oscillator arrays, systems, computer readable media, and related methods for determining binding kinetics of ligands with membrane proteins. In some embodiments, for example, the methods and related aspects of the present disclosure address pre-existing challenges of measuring ligand-membrane protein binding activities via the use of virion oscillators. In some of these embodiments, human GPCRs are displayed on the viral envelopes of human herpes simplex virus-1 (HSV-1), which removes the need of extraction, purification, and reconstitution of the transmembrane proteins. In these embodiments, each virion is then typically tethered to a sensor chip with a flexible polymer linker to form an oscillator. By applying an alternating electric field to the chip, the virion oscillates, and the oscillation amplitude is tracked in real-time with sub-nanometer precision using a plasmonic imaging technique. Upon binding of ligands or drugs to the GPCRs on the virion envelopes, the oscillation amplitude changes, from which binding kinetics and affinity are quantified. These and other aspects will be apparent upon a complete review of the present disclosure, including the accompanying figures.

In one aspect, the present disclosure provides a method of determining binding kinetics of a ligand (e.g., an antibody, a small molecule, or the like). The method includes contacting the ligand with a first surface of a substrate, which first surface comprises an electrically conductive coating and a population of virions connected to the first surface via one or more linker moieties, wherein viral envelopes of the virions display one or more proteins that bind, or are capable of binding, to the ligand, applying an alternating current electric field to the substrate to induce the virions to oscillate proximal to the first surface of the substrate, and detecting changes in oscillation amplitudes of the virions over a duration, thereby determining the binding kinetics of the ligand.

In some embodiments, the method includes detecting the changes in the oscillation amplitudes of the virions using a plasmonic imaging technique and/or a microscopic imaging technique. In some embodiments, the electrically conductive coating comprises gold (Au), indium tin oxide (ITO), silver (Ag), copper (Cu), and/or aluminum (Al). In some embodiments, the linker moieties comprise polyethylene glycol (PEG) moieties and/or biomolecules (e.g., a nucleic acid, a protein, or the like). In some embodiments, one or more spacer moieties are connected to the first surface and/or to the linker moieties. In some embodiments, wherein the virions comprise human herpes simplex virus-1 (HSV-1) virions. In some embodiments, the proteins comprise a G-protein-coupled receptor (GPCR). In some embodiments, the method further includes transfecting the virions with nucleic acid vectors that encode the proteins prior to the contacting step. In some embodiments, the method includes quantifying the binding kinetics and binding affinity of the ligand using the detected changes in the oscillation amplitudes of the virions over the duration. In some embodiments, the method includes determining the binding kinetics of the ligand in substantially real-time.

In some embodiments, the detecting step comprises introducing an incident light toward a second surface of the substrate to induce a plasmonic wave at least proximal to the first surface of the substrate and detecting a change in intensity of the incident light reflected at an interface of the first surface of the substrate. In some embodiments, the method includes introducing the incident light via at least one objective lens and/or at least one prism. In some embodiments, the method includes introducing the incident light using a superluminescent diode (SLED). In some embodiments, the method includes detecting the changes in the oscillation amplitudes of the virions over the duration using a CMOS camera.

In another aspect, the present disclosure provides a virion oscillator array device, comprising a substrate that comprises a first surface that comprises an electrically conductive coating and a population of virions connected to the first surface via one or more linker moieties, wherein viral envelopes of the virions display one or more proteins that bind, or are capable of binding, to a ligand. In some embodiments, the electrically conductive coating comprises gold (Au), indium tin oxide (ITO), silver (Ag), copper (Cu), and/or aluminum (Al). In some embodiments, the linker moieties comprise polyethylene glycol (PEG) moieties and/or biomolecules (e.g., a nucleic acid, a protein, or the like). In some embodiments, one or more spacer moieties are connected to the first surface and/or to the linker moieties. In some embodiments, the virions comprise human herpes simplex virus-1 (HSV-1) virions. In some embodiments, the proteins comprise a G-protein-coupled receptor (GPCR).

In another aspect, the present disclosure provides a system for determining binding kinetics of a ligand. The system includes a substrate having a first surface and a second surface opposite the first surface, wherein the first surface comprises an electrically conductive coating and a population of virions connected to the first surface via one or more linker moieties, wherein viral envelopes of the virions display one or more proteins that bind, or are capable of binding, to the ligand, a power source electrically connected to the substrate, which power source is configured to apply an alternating current electric field to the substrate, and an objective lens or a prism disposed proximal to the second surface of the substrate. The system also includes a light source configured to introduce light through the objective lens or the prism to induce a plasmonic wave at least proximal to the first surface of the substrate, and a detector configured to collect light reflected from the substrate. In addition, the system also includes a controller that comprises, or is capable of accessing, computer readable media comprising non-transitory computer-executable instructions which, when executed by at least one electronic processor, perform at least: applying an alternating current electric field to the substrate to induce the virions to oscillate proximal to the first surface of the substrate using the power source; introducing an incident light toward the second surface of the substrate from the light source to induce the plasmonic wave at least proximal to the first surface of the substrate; and detecting changes in oscillation amplitudes of the virions over a duration to thereby determine the binding kinetics of the ligand.

In another aspect, the present disclosure provides a computer readable media comprising non-transitory computer executable instruction which, when executed by at least electronic processor, perform at least: applying an alternating current electric field to a substrate having a first surface and a second surface opposite the first surface, wherein the first surface comprises an electrically conductive coating and a population of virions connected to the first surface via one or more linker moieties, wherein viral envelopes of the virions display one or more proteins that bind, or are capable of binding, to a ligand, which alternating current electric field induces the virions to oscillate proximal to the first surface of the substrate using the power source; introducing an incident light toward the second surface of the substrate from the light source to induce the plasmonic wave at least proximal to the first surface of the substrate; and detecting changes in oscillation amplitudes of the virions over a duration to thereby determine the binding kinetics of the ligand.

In some embodiments of the system or computer readable media, the electrically conductive coating comprises gold (Au), indium tin oxide (ITO), silver (Ag), copper (Cu), and/or aluminum (Al). In some embodiments of the system or computer readable media, the linker moieties comprise polyethylene glycol (PEG) moieties and/or biomolecules (e.g., a nucleic acid, a protein, or the like). In some embodiments, one or more spacer moieties are connected to the first surface and/or to the linker moieties. In some embodiments of the system or computer readable media, the virions comprise human herpes simplex virus-1 (HSV-1) virions. In some embodiments of the system or computer readable media, the proteins comprise a G-protein-coupled receptor (GPCR). In some embodiments of the system or computer readable media, the non-transitory computer-executable instructions which, when executed by the electronic processor, further perform at least: quantifying the binding kinetics and binding affinity of the ligand using the detected changes in the oscillation amplitudes of the virions over the duration.

### BRIEF DESCRIPTION OF DRAWINGS

FIG. 1 is a flow chart that schematically shows exemplary method steps of determining binding kinetics of a ligand according to some aspects disclosed herein.

FIGS. 2A-2D. VirD-Oscillator microarray detection technology for measuring ligand binding to GPCRs in their native membrane environment. a) Herpesvirus virions are transfected to display human GPCRs (derived from human cellular Golgi membrane) on their envelops; b) The virions are tethered to a gold sensing chip via flexible PEG linkers to form a virion oscillator array; c) Each virion is driven into oscillation with an alternating electrical field and the oscillation amplitude is detected by a plasmonic imaging technique with sub-nm precision; d) Molecular binding to the GPCRs on the virion change the oscillation amplitude (due to changes in the charge of the virions), which allows determination of the binding kinetics.

FIGS. 3A-3D. Measuring VirD oscillation amplitude with plasmonic imaging. a) Plasmonic image of single virion-oscillators, each showing a parabolic pattern (arising from the scattering of surface plasmonic waves by the virion). b) Snapshots of one virion-oscillator during different phases of an oscillation cycle, where the image intensity change reflects the change in virion-substrate distance (and thus the oscillation amplitude). c) Virion-substrate distance (red) of the virion-oscillator in (b) and applied field (blue) with frequency of 5 Hz. d) Fast Fourier Transform (FFT) of oscillation amplitude (red) showing a pronounced peak at 5 Hz, and the peak amplitude is the oscillation amplitude, where the black line is from the control obtained by performing FFT without applied electric field.

FIG. 4 illustrates the production of 315 human non-odorant VirD GPCRs.

FIG. 5. Antibody specificity tests on VirD GPCRs. The VirD GPCR array was fabricated by spotting down 100 pL of each purified virion in duplicate on an epoxy-grafted glass slide. The quality of the array was examined with anti-gD mAb. Listed are 20 commercial mAbs raised against the ecotodomain of human GPCRs and their binding specificity was tested individually on the array. A highly specific anti-CXCR2 mAb is shown in the middle panel as an example.

FIGS. 6A-6D. Measuring small molecule binding to GPCR with VirD-Oscillators. a-c) Binding kinetic curves (oscillation amplitude changes vs. time) of three ligand/VirD GPCR pairs, where the solid lines are global fitting of the data to the first order kinetics. Each binding curve is average of at least 5 virions. Ligand structure, kinetic and equilibrium constants are labeled next to the graphs. d) Negative control experiment performed on virions without GPCRs displayed shows no binding to any of the small molecule ligands. Applied voltage: 0.4 V at 5 Hz. Buffer: diluted PBS buffer at pH 7.4.

FIGS. 7A-7D. Validation of binding specificity with fluorescence detection. a) Bright field and fluorescence images of virions with displayed GPCRs. The bright field images reveal individual virions (bright spots), and the fluorescence images obtained without/with the ligands confirm the specific binding of the ligands to the GPCRs on the virions. b) Negative control: none of the off-target ligands generate observable fluorescence changes on K082. c) Fluorescent intensity of the virions with/without the ligands. d) No measurable fluorescent on K082 virions (GPCR free) with the ligands. The dashed lines in c and d indicate background fluorescence levels.

FIGS. 8A-8D. Measuring binding kinetics with VirD-Oscillator arrays using wide-view plasmonic microscopy. a) VirD-Oscillators spotted on a gold chip and imaged with a low magnification (wide-view) plasmonic imaging setup. Although the individual VirD-Oscillators cannot be resolved, the image intensity of each spot oscillates with the applied field provides the average oscillation amplitude of the VirD-Oscillators in the spot. The red line in the plot is the image intensity from the background regions, reflecting charging effect of the gold chip, which was subtracted out in data analysis. b) Kinetics of a peptide ligand (SPRIF14 MW=1560 Da) binding to its target GPCR (SSTR2). c-d) Kinetics of SPRIF14 binding to two off-target GPCRs (GABBR1 and NTSR1). The solid lines are global fitting of the data to the first order kinetics. Applied voltage: 0.3 V amplitude at 5 Hz. Buffer: diluted PBS.

FIGS. 9A-9B. Wide-view plasmonic imaging of a protein microarray. a) Plasmonic image showing the binding of anti-BSA antibody at different concentrations to a 10×10 protein microarray of BSA. The microarray dimensions are 8×8 mm, and the space between spots is 0.8 mm. b) Selected binding kinetic curves (grey) and fittings (colored) of anti-BSA at different concentrations. For clarity, only a fraction of the 100 recorded curves are shown here.

FIG. 10 schematically shows exemplary steps to fabricate a VirD-Oscillator microarray according to some aspects disclosed herein.

FIG. 11. DNA-coded VirD-Oscillator microarray. Production of gold chips pre-spotted with thiolayted double-stranded DNAs terminated with short single stranded DNAs, and virion functionalized with complementary short single stranded DNAs, both can be long-term stored in a freezer and shipped to end user. Prior to experiment, the VirD-Oscillator microarray can be assembled by the user via one step DNA hybridization.

FIG. 12. Exemplary plasmonic VirD-Oscillator microarray detection system (microarray reader). A 690 nm laser beam is expanded and collimated on to a glass prism at SPR angle. The reflected beam is focused into a fast CMOS imager (camera 1) via a variable zoom lens set to produce SPR image of the whole array (a). The VirD-Oscillator chip is mounted onto the prism. A flow cell with integrated electrodes is placed on top of the sensor chip. An alternating electrical potential is applied between the gold chip and embedded counter electrode to drive the VirD-Oscillators into oscillation. A high magnification objective is mounted on top of the flow cell to collect high-resolution scattered SPR image (b) from individual VirD-Oscillators in a selected array spot via a second CMOS imager (camera 2).

FIG. 13. Known ligands of non-odorant GPCRs.

FIG. 14. Validation of off-targets of anti-ACRK3. Left: Immunofluorescence assays performed with anti-ACKR3 on intact Vero cells infected with viruses carrying ACKR3, CALCR, or GPR61. Anti-ACKR3 mAb shows strong staining to all three infected cells compared with the negative control cells infected with K082, suggesting that the mAb can recognize CALCR and GPR61 embedded in intact cell membranes. Right: Immunoblot (IB) analysis of anti-ACKR3 against cell lysates of these infected cells under either native or denatured conditions also show strong recognition of all three GPCRs.

FIGS. 15A-15H. Plasmonic scattering microscopy for detection of single virion oscillators. A) A picture of testing setup. B) A schematic drawing of the flow cell. C) Original image gets from top objective. D) Differential image of adjacent frames. E-G) Statistics of intensity from 26 nm, 44 nm and 65 nm polystyrene particles. H) Calibration curve between size and intensity.

FIGS. 16A-16F. Single protein oscillator detection. A) Fast Fourier Transform results at different applied voltage. B) Single oscillation curves selected from circled spots in A. C-E) Plateau intensity of single oscillator with different tether lengths. 3.4K, 5K and 10K PEG linker and 50 nm streptavidin coated polystyrene nanoparticles in this case. F) The intensity changes in evanescent field for each length of PEG linker.

FIGS. 17A-17E. Storage test for VirD-oscillator. A) Workflow and conditions for virion oscillator storage test. Oscillators were fabricated to a sensor chip and the buffer was replaced with 50% glycerol, which can protect the protein molecules and keep the buffer in liquid state at −20° C. Then the chips were placed into a small box and filled with nitrogen to reduce the oxidation rate. Finally, the box was sealed and stored in a freezer. B) Test results of fresh oscillator sample. C-E) Test results with different storage time.

FIGS. 18A-18D. Large view area plasmonic imaging system with electrochemical flow cell. A) Optical diagram and B) Picture of the plasmonic imaging system use for virion oscillator array detection. C) Schematic of the flow cell design and D) The connection of electrodes and tubing of the flow cell in actual measurement. WE: working electrode, CE: counter electrode, RE: reference electrode.

FIG. 19 schematically shows an exemplary workflow for fabricating a virion oscillator microarray on gold sensor chip steps according to some aspects disclosed herein.

FIGS. 20A-20-E. Ligand binding measurement and fitting results from single type virion oscillator array demonstration. A-C) Oscillation amplitude change of GPCR virion (CHRM3, ADRB2 and NTSR1 respectively) oscillator to corresponding ligands. D) Kinetics and equilibrium constants of ligand binding. E) SPR image of a virion oscillator array with different virions.

FIG. 21 is a schematic diagram of an exemplary system suitable for use with certain aspects disclosed herein.

### DEFINITIONS

In order for the present disclosure to be more readily understood, certain terms are first defined below. Additional definitions for the following terms and other terms may be set forth throughout the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

It is also to be understood that the terminology used herein is for the purpose of describing particular embodiments only and is not intended to be limiting. Further, unless defined otherwise, all technical and scientific terms used herein have the same meaning as commonly understood by one of ordinary skill in the art to which this disclosure pertains. In describing and claiming the methods, systems, and computer readable media, the following terminology, and grammatical variants thereof, will be used in accordance with the definitions set forth below.

About: As used herein, “about” or “approximately” or “substantially” as applied to one or more values or elements of interest, refers to a value or element that is similar to a stated reference value or element. In certain embodiments, the term “about” or “approximately” or “substantially” refers to a range of values or elements that falls within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value or element unless otherwise stated or otherwise evident from the context (except where such number would exceed 100% of a possible value or element).

Antibody: As used herein, the term “antibody” refers to an immunoglobulin or an antigen-binding domain thereof. The term includes but is not limited to polyclonal, monoclonal, monospecific, polyspecific, non-specific, humanized, human, canonized, canine, felinized, feline, single-chain, chimeric, synthetic, recombinant, hybrid, mutated, grafted, and in vitro generated antibodies. The antibody can include a constant region, or a portion thereof, such as the kappa, lambda, alpha, gamma, delta, epsilon and mu constant region genes. For example, heavy chain constant regions of the various isotypes can be used, including: IgG₁, IgG₂, IgG₃, IgG₄, IgM, IgA₁, IgA₂, IgD, and IgE. By way of example, the light chain constant region can be kappa or lambda. The term “monoclonal antibody” refers to an antibody that displays a single binding specificity and affinity for a particular target, e.g., epitope.

Biomolecule: As used herein, “biomolecule” refers to an organic molecule produced by a living organism. Exemplary biomolecules, include without limitation macromolecules, such as nucleic acids, proteins, peptides, oligomers, carbohydrates, and lipids.

Ligand: As used herein, “ligand” refers to a substance that forms a complex with another molecule, such as a biomolecule.

Nucleic Acid: As used herein, “nucleic acid” refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids can also include nucleotide analogs (e.g., bromodeoxyuridine (BrdU)), and non-phosphodiester internucleoside linkages (e.g., peptide nucleic acid (PNA) or thiodiester linkages). In particular, nucleic acids can include, without limitation, DNA, RNA, cDNA, gDNA, ssDNA, dsDNA, cfDNA, ctDNA, or any combination thereof.

Protein: As used herein, “protein” or “polypeptide” refers to a polymer of at least two amino acids attached to one another by a peptide bond. Examples of proteins include enzymes, hormones, antibodies, and fragments thereof.

### DETAILED DESCRIPTION

Membrane proteins, such as G-protein-coupled receptors (GPCRs), are responsible for many cellular functions, and are an important family of drug targets, but they are extremely difficult to study with pre-existing technologies, because GPCRs are multi-pass transmembrane receptors, which need native cellular membranes to maintain their conformations and functions for affinity and kinetic measurement. In some aspects, the present disclosure overcomes these challenges by providing a virion oscillator microarray technology to measure molecular binding to GPCRs, among other membrane proteins. The present disclosure thus provides a long-sought tool to determine cancer-related cellular signaling processes, screen drugs targeting membrane proteins, and validate new therapies for cancer and other diseases, among other aspects.

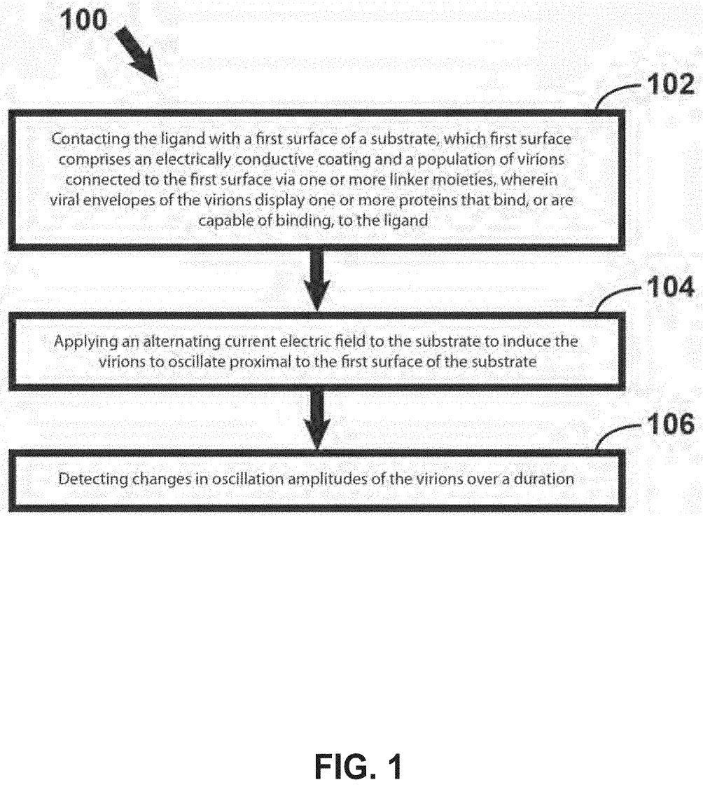

By way of further overview, FIG. 1 is a flow chart that schematically shows exemplary method steps of determining binding kinetics of ligands according to some aspects disclosed herein. As shown, method 100 includes contacting the ligand with a first surface of a substrate, which first surface comprises an electrically conductive coating and a population of virions connected to the first surface via one or more linker moieties in which viral envelopes of the virions display one or more proteins that bind, or are capable of binding, to the ligand (step 102). Method 100 also includes applying an alternating current electric field to the substrate to induce the virions to oscillate proximal to the first surface of the substrate (step 104). In addition, method 100 also includes detecting changes in oscillation amplitudes of the virions over a duration to thereby determining the binding kinetics of the ligand (step 106).

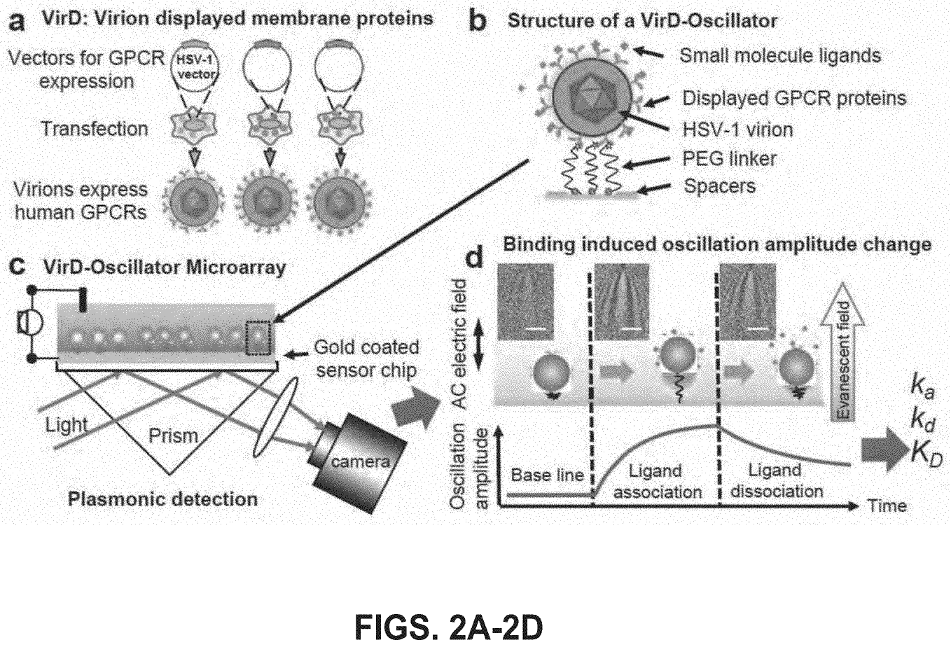

In some embodiments, a Virion Display (VirD)-Oscillator microarray detection platform is provided. Using the VirD technology, human GPCRs are displayed on the human herpes simplex virus-1 (HSV-1) envelope (FIG. 2a). The HSV-1 envelope is derived from the human cellular Golgi membrane, and thus preserves the native conformations and post-translational modifications of the transmembrane proteins. Each virion is then tethered via a flexible polymer to a sensor chip (gold-coated glass slide; see FIG. 2b). Upon applying an alternating electric field to the chip, the virion oscillates, and the oscillation amplitude is detected by a plasmonic imaging technique with sub-nanometer precision (FIG. 2c). Binding of a ligand or drug molecule to the GPCR displayed on the virion causes a change in the oscillation amplitude, which is measured in real-time to provide binding kinetics and affinity without the need of extracting the transmembrane protein from its native membrane environment, and also without the need of labelling the ligands (FIG. 2d). This technology removes the tedious steps required to extract and purify multi-pass transmembrane proteins, overcomes the stability issue associated with extraction, purification and reconstitution, eliminates labelling and associated adverse effects, and allows quantifying the kinetics of large and small molecule binding to the proteins.

As described further herein, a VirD collection of 315 non-odorant human GPCRs has been developed, which covers 85% of all annotated non-odorant GPCRs in humans. These non-odorant GPCRs are desired drug targets and are studied to further validate the technology disclosed herein. Other transmembrane proteins are also optionally adapted for use with the methods and other aspects of the present disclosure. Some embodiments provide a VirD-Oscillator microarray with 315 non-odorant human GPCRs on a single chip. In addition to microarrays, VirD-Oscillator microarray readers for quantifying ligand binding to GPCRs are also provided. The VirD-Oscillator microarrays and readers can be used to measure the kinetics of small and large ligand binding with cancer related human GPCRs and other transmembrane proteins.

Importance of GPCRs in cancer mechanisms and therapeutics

Membrane proteins relay signals between a cell and its external environment, transport ions and molecules in and out of the cell, and allow the cell to recognize and interact with other cells. They are also the most popular drug targets, accounting for over a half of the FDA approved drugs. A particularly important family of human transmembrane proteins is G-protein-coupled receptors (GPCRs), which constitute the largest and most diverse transmembrane protein family in the human genome with over 800 members identified to date. GPCRs play important roles in numerous cellular and physiological processes, including cell proliferation, differentiation, neurotransmission, development and apoptosis, and cancer growth and development. Unsurprisingly, over 30% of FDA-approved drugs target this family. The recent advances in crystallography has led to structural determination for an ever-increasing number of GPCRs, which has stimulated a surge of efforts to develop drugs targeting GPCRs for various diseases, including cancer. GPCRs are considered to be the most useful drug targets of many solid cancers. Tumor cell proliferation is regulated by many neuropeptides, and signaling molecules, such as prostaglandin E2, thrombin, sphingosine-1-phosphate, lipoprotein A and interleukin 8, which often target GPCRs to initiate the downstream signaling networks that involve second messenger-generating systems, small GTPases of the Ras and Rho families, and MAPK cascades. A review article, for example, listed more than 47 non-odorant GPCRs in 13 different cancers. For example, beta-2 adrenergic receptor (ADRB2) is involved in regulating growth and/or metastasis in non-small-cell lung cancer.

Difficulties in Studying GPCRs in their Native Membrane Environment

Understanding the vital cellular functions and screening new cancer drugs targeting GPCRs require measuring of the binding of ligands and drugs with the GPCRs. Despite the importance, developing such a capability has been challenging. Traditional methods use radiolabelled or fluorescent-labelled ligands. These technologies are end-point measurements, which provide affinity but not binding kinetics. Binding kinetics is critical for determining drug efficacy, residence time and biased agonism, and for elucidating ligand-target binding mechanism in drug design. To determine molecular binding kinetics, label-free detection technologies have been developed. However, these technologies usually involve extracting proteins from cells, followed by purifying the extracted proteins and immobilizing them on a sensor surface for binding measurement. This approach unfortunately cannot be directly applied to GPCRs and other multi-pass transmembrane proteins, because these proteins unavoidably lose their native conformations and functions outside the membranes.

Several methods have been developed to prepare and stabilize transmembrane proteins for binding kinetic studies. Examples includes inserting the proteins in artificial membrane bilayers, such as lipid vesicles and nanodisc, and cell membrane preps. We have developed a method to directly measure membrane protein binding kinetics on whole cells. Despite the success, high throughput quantification of ligand or drug binding with a large collection of GPCRs remains challenging. This is especially the case for measuring small molecule binding to the proteins. Small molecules are difficult to detect and quantify because the existing label-free detection technologies typically detect the relative mass changes, thus difficult for small molecules. Small molecule drugs are prevalent for transmembrane proteins, and account for ˜90% of the FDA approved drugs. A capability of detecting small molecule binding to GPCRs in their native membrane environment in a high-throughput fashion is thus needed to advance the study of GPCRs and develop new drugs targeting the transmembrane proteins. None of the existing methods can meet these requirements (Table 1).

TABLE 1
Comparison of VirD with other membrane protein preparation methods

| | Methods | | | |
|---|---|---|---|---|
| | Membrane | Nano- | Whole | |
| Capability | Prep | disc | cell | VirD |
| Maintain native cellular | Partial* | Partial* | Yes | Yes |
| environment | | | | |
| Allow measure small molecule | No | Yes ** | Yes *** | Yes |
| binding kinetics | | | | |
| Enable study of large GPCR | No | No | No | Yes |
| collections | | | | |

Notes:
*Although membrane preps and nanodiscs mimic lipid environment, they lack the separate intra and extra cellular environment.
**Nanodisc stabilized membrane proteins has been studied by us with a charge sensitive optical detection method.
***Binding kinetics of whole cells has been demonstrated by us via tracking mechanical deformation of the cells. These technologies fall short for rapid quantification of a large collection of transmembrane proteins in their native membranes.

VirD-Oscillator Microarray Detection Technology

To address the unmet need, we disclose herein a virion-displayed (VirD) oscillator microarray and detection technology that integrates the VirD technology for multipass GPCRs with an innovative VirD-Oscillator plasmonic detection platform to achieve label-free and real-time kinetic measurements of binding between small molecule ligands and GPCRs. As illustrated in FIG. 2, GPCRs are displayed in HSV-1 envelopes derived from human Golgi membranes, which preserve the native conformations and post-translational modifications of the GPCRs. Each virion with a GPCR displayed is tethered to a sensor chip (gold-coated glass slide) via a flexible polymer linker. By applying an alternating electric field to the gold surface, the virion is driven into oscillation and the oscillation amplitude is monitored using a plasmonic imaging technique with sub-nm precision. When ligand molecules bind to the GPCRs on the virion, the oscillation amplitude changes, which is measured in real-time to provide precise binding kinetics.

Extensive experiments have been conducted to demonstrate the VirD-Oscillator microarray and detection technology for measuring the binding of human GPCRs with small molecule ligands. As described further herein, we perform key experiments to transform the technology into a powerful high-throughput platform for quantifying small molecule and antibody binding kinetics with 315 non-odorant human GPCRs on a single VirD-Oscillator microarray chip.

VirD: A Virion Display Technology for Displaying Membrane Proteins in their Native Membranes

We have developed a VirD technology for profiling functional membrane proteins. VirD uses recombinant HSV-1 to display human GPCRs on the viral envelopes by replacing a gene expressing the major viral envelope glycoprotein B (gB) with a GPCR ORF (FIG. 2a). The HSV-1 envelope is derived from the human Golgi membranes to preserve the native conformations and post-translational modifications of the membrane proteins. The VirD technology eliminates the need of extraction, purification and reconstitution of the membrane proteins, which alleviates the aforementioned difficulties in the existing methods. VirD have been developed for 315 non-odorant human GPCRs, which covers 85% of all annotated non-odorant human GPCRs as preferred drug targets. The remaining challenge was to measure the affinity and kinetics of ligand molecules and drug candidates binding with the GPCRs in a high-throughput fashion. Each virion has a large mass (˜200 MDa) compared to a protein (˜100 kDa) and therefore, the relative mass change caused by binding to a protein ligand is small. The relative mass change associated with the binding of a small molecule ligand (˜100-1k Da) is even smaller. None of the commercial detection technologies are capable of detecting such small mass changes in the virions.

VirD-Oscillator: A Microarray Detection Technology for Quantifying the Binding of Large and Small Ligands to Transmembrane Protein on Single Virions

We developed and validated a new label-free detection technology (VirD-Oscillator), which features synergistic coupling of the virion display technology with an innovative plasmonic imaging technology to overcome the limitations of the existing technologies. It tethers individual virions to a gold sensor chip with a flexible polyethylene glycol (PEG) polymer (FIG. 2c). The virion is driven into oscillation with an alternating electric field, and the oscillation amplitude (z₀) is given by

$${z}_{θ} = \frac{{q}^{*}}{{k}^{*}} {E}_{0} , \qquad ( 1 )$$

where q* is the effective charge of the virion, k* is the effective spring constant of the polymer linker, and E₀ is the amplitude of the alternating electric field. Upon ligand binding to the target proteins on the virion, the oscillation amplitude changes. The change occurs because most biologically relevant ligands and drugs are charged or partially charged (containing charged groups). For instance, only 10 of the 2039 FDA approved small molecule drugs are uncharged. Even for uncharged ligands, the binding can still alter the charge via conformation changes of the membrane proteins, thus detectable. We have shown that both charged and uncharged molecules can be detected (see additional data described further herein). Since the VirD-Oscillator detection technology is not based on mass detection, its sensitivity does not diminish with the molecular mass of the ligand, making it suitable for studying both large and small ligands.

E₀ and k* in Eq. 1 can be determined with calibration, from which the effective charge (q*) can be quantified. However, for binding kinetics measurement, one only needs to measure the relative change in the oscillation amplitude vs. time. For the same reason, the number of the linkers to each virion will affect k* and oscillation amplitude, but not binding kinetics directly.

Sensitive Detection of Single Virion Oscillation Via Plasmonic Imaging

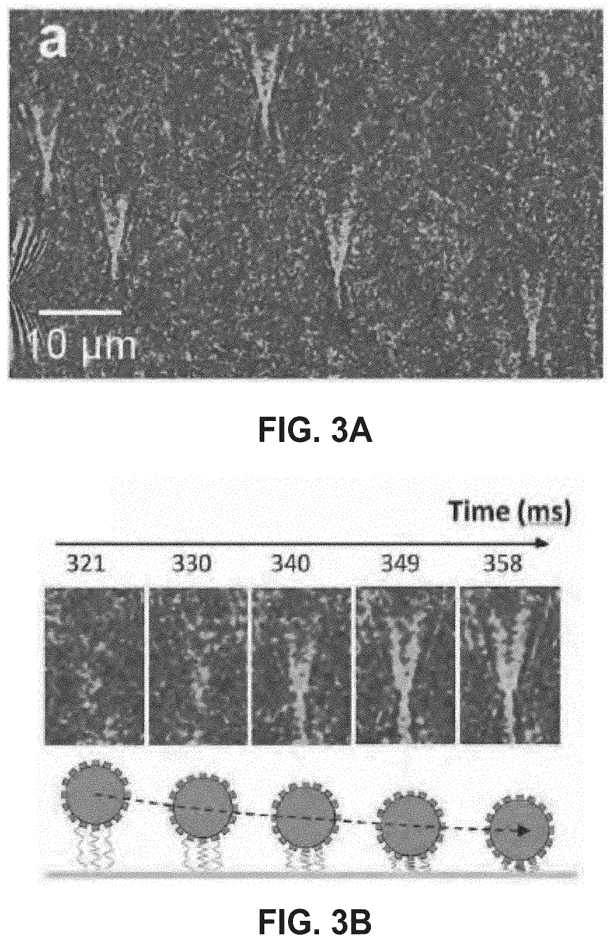

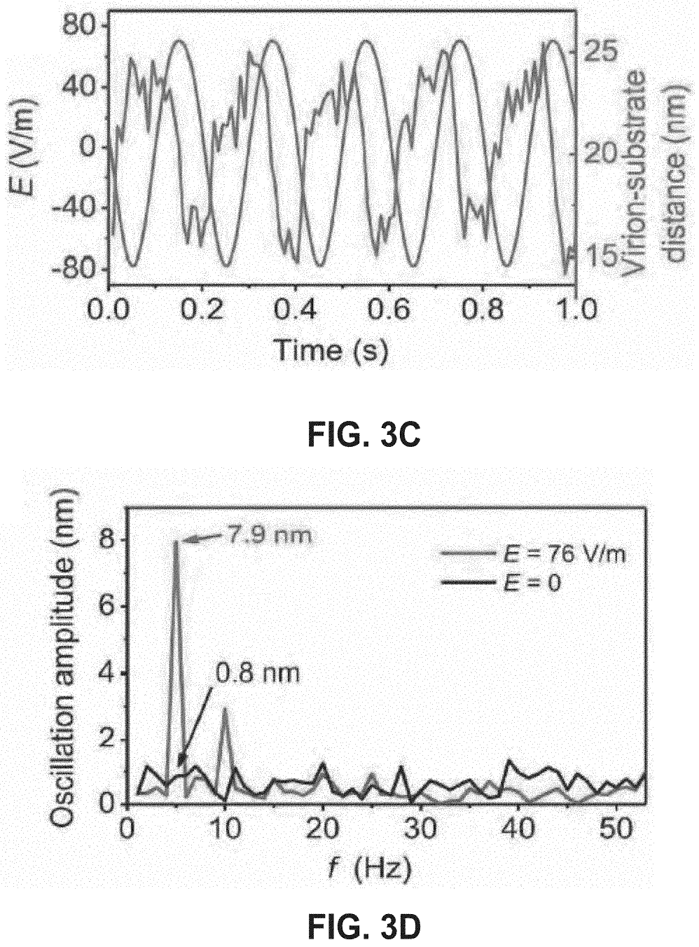

The detection limit of VirD-Oscillator microarray is determined by how accurate one can measure the oscillation amplitude (Eq. 1). Using a plasmonic imaging system, we have shown that the oscillation amplitude of a nanoparticle can be detected with sub-nm precision (FIG. 3). This extremely high sensitivity comes from the scattering of the plasmonic wave by the virion (diameter of ˜200 nm), which produces a parabolic pattern for each virion (FIG. 3a) with image intensity that decays rapidly with the distance between the virion and the chip surface (FIG. 3b). The plasmonic imaging (parabolic pattern) intensity (I) is related to the distance between the virion and the sensor surface (z), given by

ΔI/I=−z₀/100 nm (2)

where ΔI/I is the relative intensity change of the virion, and 100 nm is the decay distance of the evanescent field. Our experiments have shown that the oscillation can be tracked in real time (FIG. 3c) with a precision of ˜0.8 nm (FIG. 3d). The corresponding charge detection limit is ˜1.4e, where e=1.6×10⁻¹⁹ C, the charge of a single electron. This detection limitation is among the most sensitive detection technologies.

Simple and Robust Fabrication of VirD-Oscillator Microarray Via Self-Assembly

VirD-Oscillators can be self-assembled on a sensor chip without complicated fabrication facilities. Each virion is tethered to the chip surface via one or more flexible polymer linkers (FIG. 2b). The polymer has two linker groups at its two ends: one end binds to the gold surface, and the other end binds to the virion. One situatable choice of the linker is PEG, which was used in experiments described further herein. We describe herein details of VirD-Oscillator microarray fabrication.

Exemplary Features of the VirD-Oscillator Microarray and Detection Platform

The technology described herein has the following exemplary unique features:
Virion display of GPCRs removes the tedious protein extraction, purification and reconstitution steps, and preserves the native functions of the transmembrane proteins.
Simple fabrication of VirD-Oscillator microarray via self-assembly.
VirD provides a large collection of important GPCRs that are easily accessible and mass-producible.
Plasmonic detection allows label-free, real-time and microarray-compatible quantification of large and small molecule binding to the GPCRs on the virions.

These features enable the study of molecular interactions of GPCRs associated with cancer and other diseases, and accelerate drug development by providing quantitative binding constants (for accurate ranking of drug candidates).

Production of 315 Non-Odorant VirD GPCRs

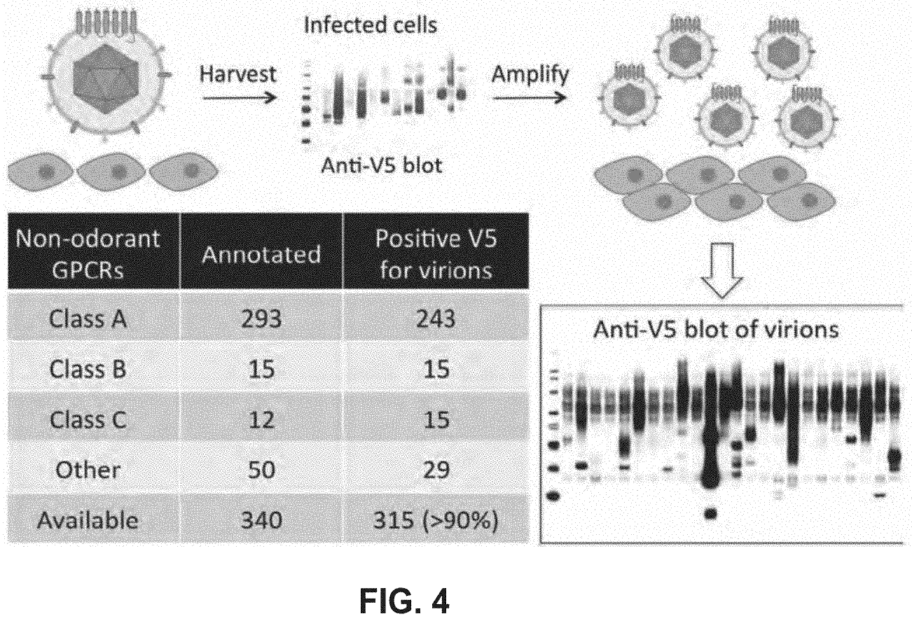

The human non-odorant GPCRs were cloned into the HSV-1 genome using the Gateway (Life, USA) method. As shown in FIG. 4, each GPCR was expressed as a viral gene under the control of the UL27 gene (the gene of gB) promoter, and the expressed polypeptide contains a C-terminal V5 epitope tag. To produce virions incorporating GPCRs, we infected Vero cells (3×10⁷ cells) at a multiplicity of infection of five plaque forming units/cell. The extracellular medium of these infected cells was collected 48 hours after infection, and then purified and concentrated via centrifugation through a 20% sucrose cushion. The final virion pellets were re-suspended in 30% glycerol and stored at −80° C. before use. Incorporation of the GPCR molecules in the virion was confirmed by immunoblot analysis using anti-V5 antibody. Of the 340 available non-odorant human GPCRs in our collection, 92% of them showed detectable anti-V5 signals.

Characterization of 315 Non-Odorant VirD GPCRs

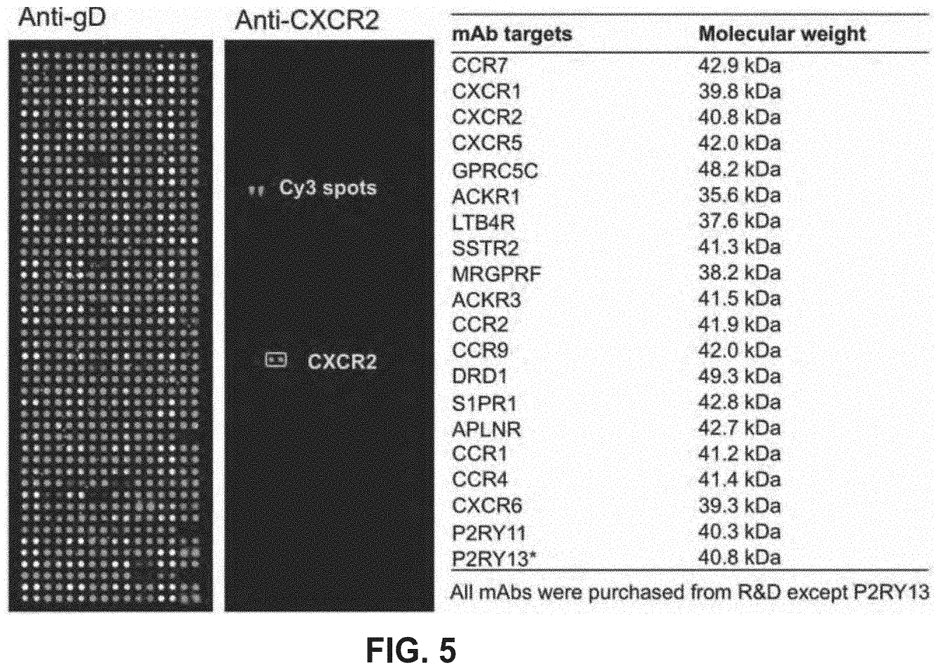

We examined antibody binding to the 315 VirD GPCRs by printing the virions on a glass slide activated with epoxy moiety, including positive (e.g., Cy3 and Cy5 dyes) and negative (e.g., K082, gB minus virion and BSA) controls using a robotic jet printer. The quality of the VirD array was examined with an anti-gD antibody (left panel; FIG. 5). All 315 GPCR virions show detectable anti-gD signals, which is consistent with the anti-V5 immunoblot analysis against purified virions, indicating the success of VirD GPCR array fabrication.

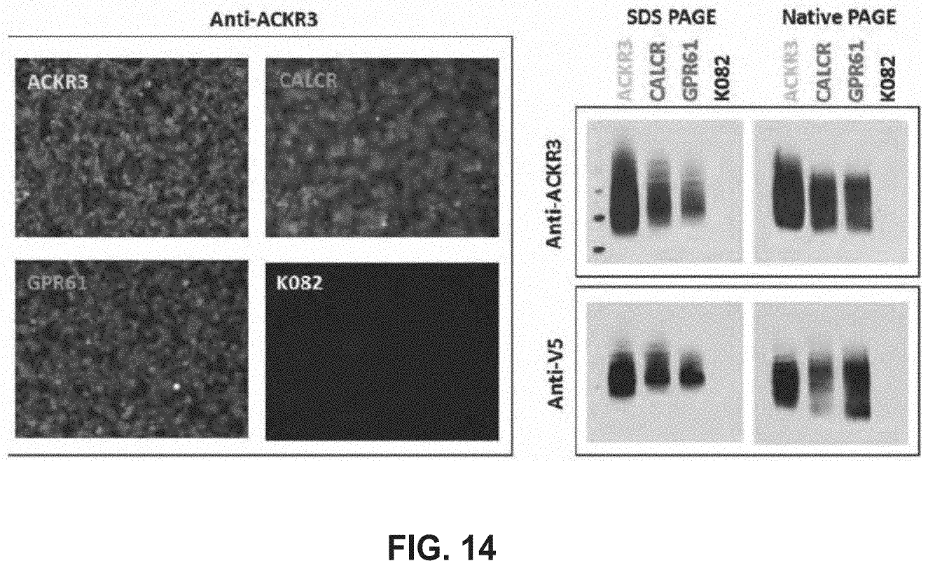

To examine the microarray ability for profiling antibody specificity, 20 monoclonal antibodies (mAbs) raised against the ectodomains of human GPCRs were used to individually probe the GPCR VirD array at a 1:2000-fold dilution. The binding signals were then detected with a Cy3-labeled secondary antibody. 10 of the 20 tested mAbs show ultra-specificity, meaning that they only recognize their intended targets without producing any off-target signals. As an example, anti-CXCR2 mAb only recognizes CXCR2 virion with a high Z-score of 20 (middle panel; FIG. 5). Anti-ACKR3 recognizes ACKR3 with a highest Z-score of 30, and two off-targets have lower Z-scores (not shown). Binding of this mAbs to ACKR3 and two off-targets, GPR61 and CALCR, was confirmed in cell surface staining of infected cells, as well as immunoblot analysis against purified virions (see FIG. 14). This result clearly indicates that these GPCRs are displayed in the expected orientations on the virions and maintain their biological functions.

Fabrication of VirD-Oscillators

We fabricated VirD-Oscillators by tethering single HSV-1 virions (˜200 nm in diameter) to a gold sensor chip via PEG (63 nm in length) (FIG. 2b). The PEG has a thiol group on one end to bind to the gold surface, and a N-hydroxysuccinimide (NHS) moiety on the other end to form a covalent bond to the virions via the primary amines of the viral glycoproteins in the envelopes. The virions were incubated with HS-PEG-NHS at 1:5 ratio in 1×PBS overnight to form a virion-PEG complex, which was diluted to 104 virions/A. 100 μL virion-PEG complex was then applied to a gold chip and incubated for 20 min to allow assembly of the virion-oscillators on the chip. The chip was then rinsed with 1×PBS, followed by incubation in 15 nM dithiolalkanearomatic PEG6-COOH overnight to passivate the exposed gold surface. The chip coated with the virion-oscillators was kept hydrated and stored at 4° C. during the modification.

Detection of Small Molecule Binding to GPCRs Displayed on Virions

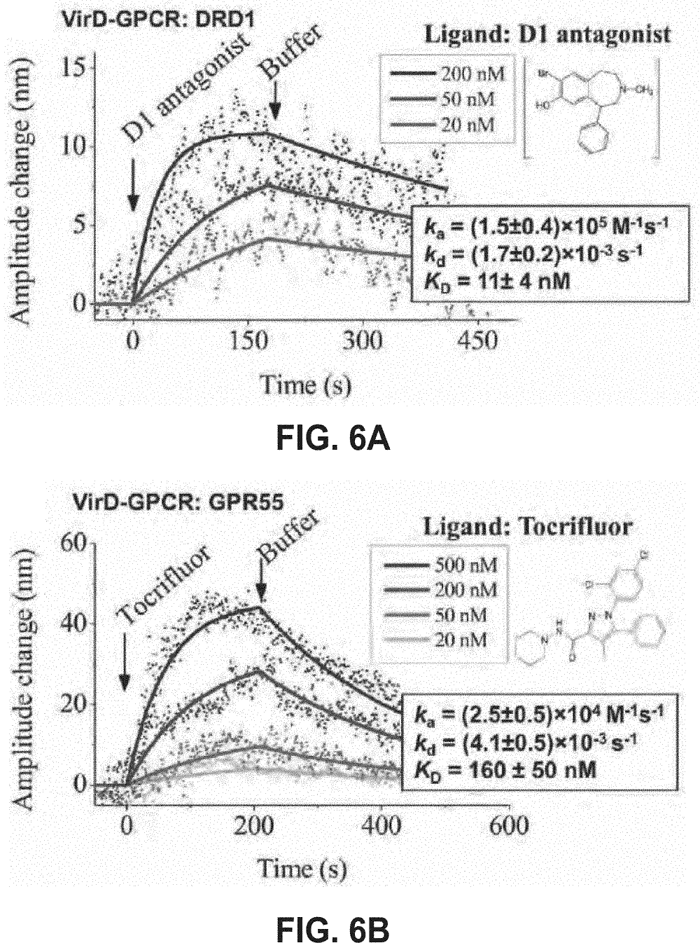

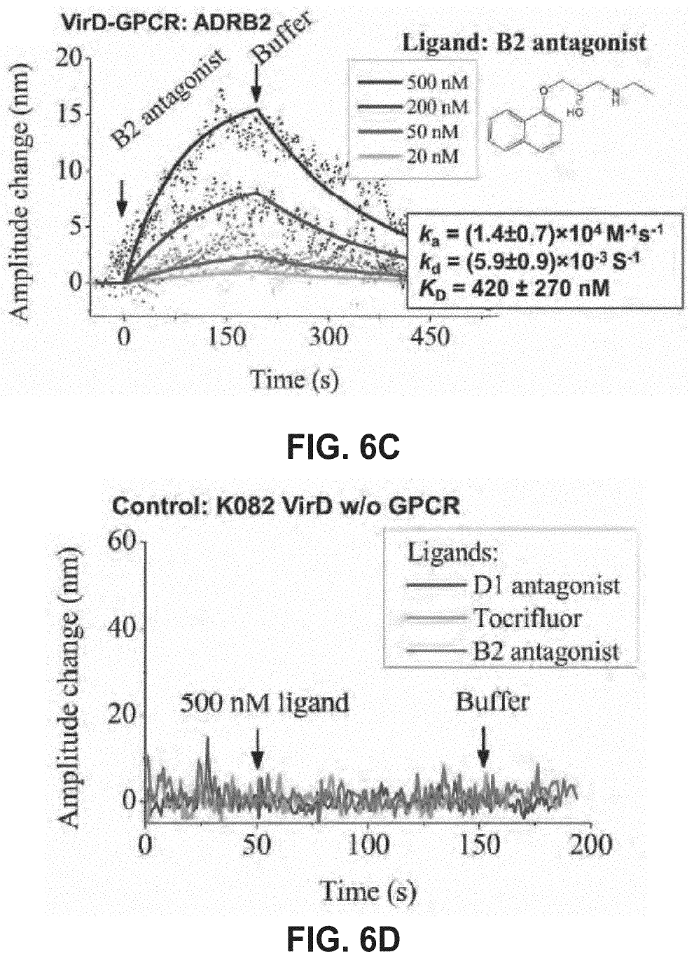

To demonstrate the capability of VirD-Oscillators, we displayed three human GPCRs (DRD1, GPR55, and ADRB2) on the HSV-1 virion envelope, fabricated virion-oscillators, and measured the binding kinetics of 3 canonical small molecule ligands (D1 antagonist, Tocrifluor, and B2 antagonist) targeting DRD1, GPR55, and ADRB2, respectively (FIG. 6). These GPCRs were chosen because they have well characterized small molecule ligands and are commercially available in both fluorescently labelled and un-labelled forms. To drive the virions into oscillation, we applied a periodic potential with amplitude, 0.4 V, and frequency, 5 Hz, to the gold chip. Initially, we flew 4 mM PBS buffer at a rate of 300 μL/min over the gold chip, and recorded the oscillation amplitude to establish a baseline, and then introduced each of the three ligands to allow binding (association) to the corresponding target GPCR on the virions. The binding induces a change in the oscillation amplitude, indicating changes in the virion effective charge associated with the ligand binding to the GPCRs.

After measuring binding, we studied unbinding of the small molecule ligands from the GPCRs by flowing PBS buffer over the chip and observed that the oscillation amplitude returned to the pre-binding level. By repeating the measurement at different concentrations of each ligand, we obtained binding curves for D1 antagonist-DRD1, Tocrifluor-GPR55, and B2 antagonist-ADRB2 binding pairs (FIGS. 6a-c). By fitting the binding curves at different concentrations globally with the first order kinetics model, we determined the association rate constant, kₐ, dissociation rate constant k_{d}, and the equilibrium constant K_{D}. Note that at pH 7.4, D1 antagonist and B2 antagonist are positively charged but Tocrifluor is neutral. These results demonstrate the VirD-Oscillator can measure both charge and uncharged molecules.

Control Experiments

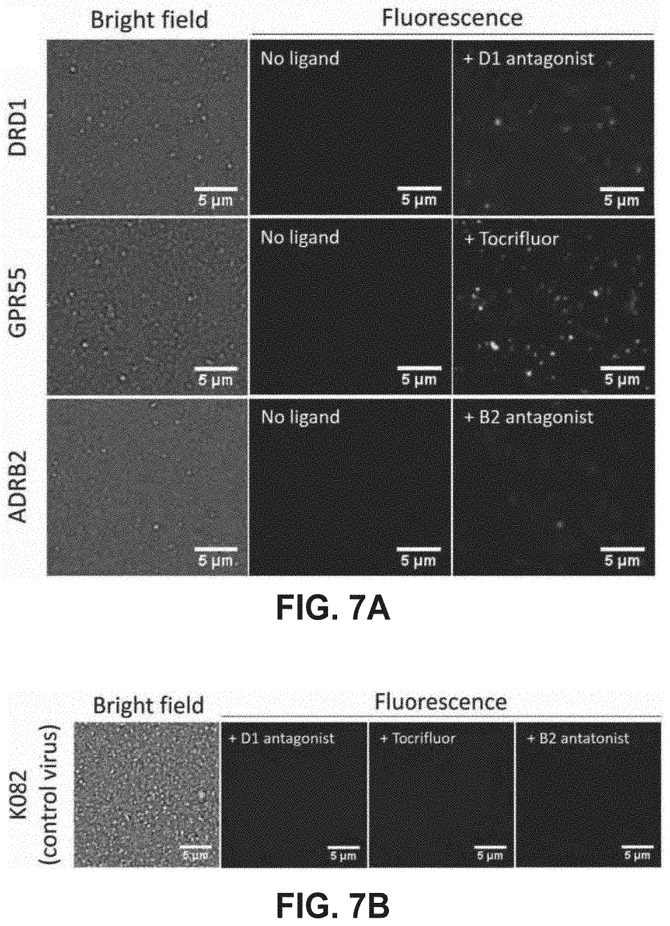

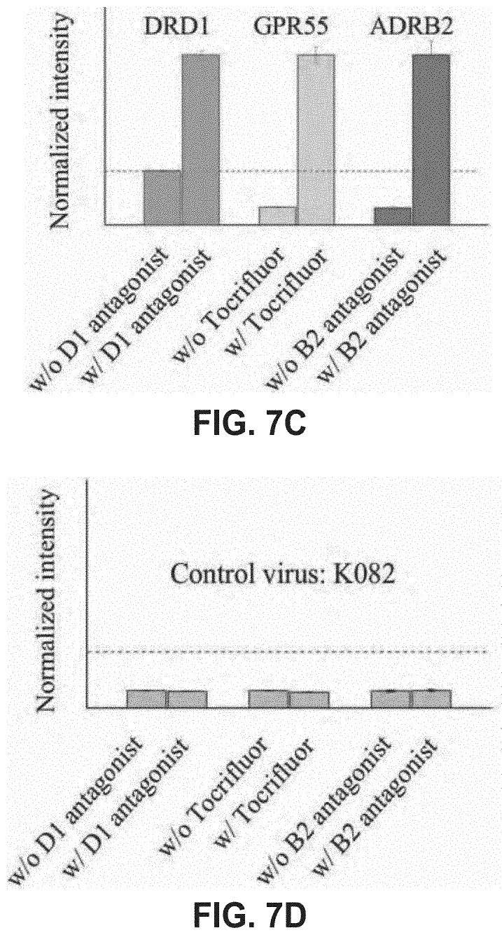

To confirm that the above results were not due to non-specific binding, we used K082, a gB null HSV-1 virion with no GPCR displayed on the envelope, as a negative control and measured its binding with each of the three ligands. We did not observe any detectable changes in the oscillation amplitude (FIG. 6d), indicating no non-specific binding of the ligands to HSV-1 virions. As a further validation of the results, we measured the binding of fluorophore-labelled small molecule ligands to the virion-oscillators with fluorescence imaging. The fluorescence shows significant increase after incubation with the corresponding ligands, and the fluorescence intensity correlates well with the oscillation amplitude change (FIGS. 7a and c). We also measured fluorescence before and after introducing off-target ligands and observed no fluorescence emission from the virions (FIGS. 7a and b). These fluorescence experiments confirmed that the results by the VirD-Oscillator detection technology were indeed due to the specific binding of each ligand to its corresponding GPCR.

High-Throughput VirD-Oscillator Detection with Wide-View Plasmonic Imaging

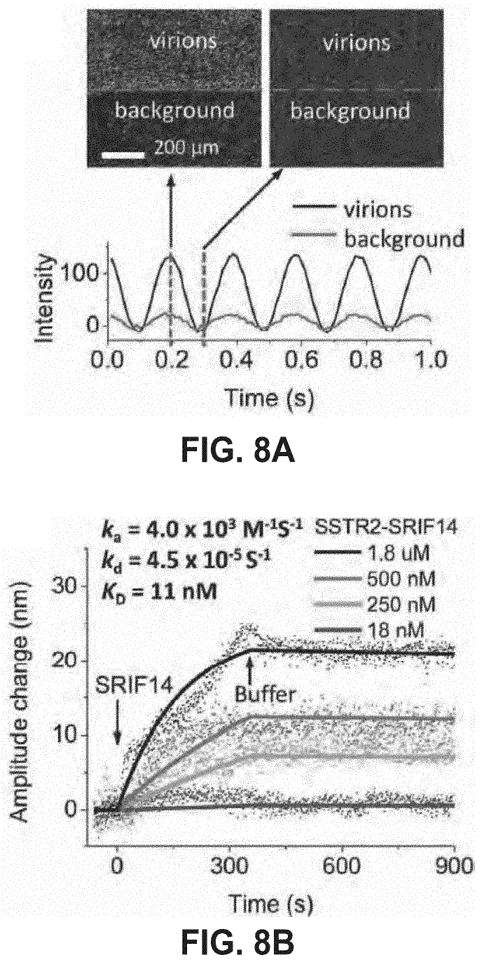

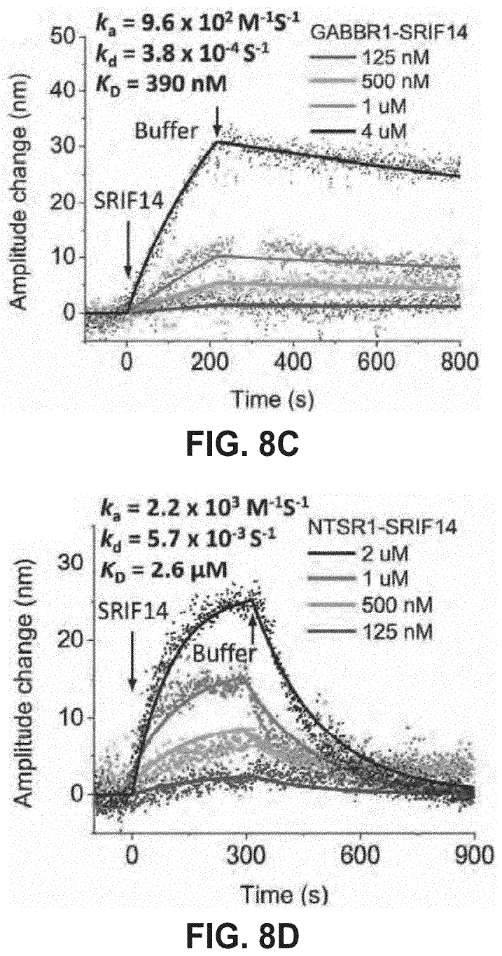

The plasmonic imaging technique can resolve and detect the binding on each of the VirD-Oscillators (FIG. 3a) for detailed examination of individual virions. This was achieved with high magnification objectives (e.g., 60×) that have relatively small view size (detection area). For high-throughput applications, e.g., simultaneous study of 315 human GPCRs on a single chip, the imaging view area must be sufficiently large for hundreds of spots (in a microarray format), which requires wide-view plasmonic imaging capability. To meet this requirement, we built a low magnification (<10×) but wide-view plasmonic imaging capability. To demonstrate this wide-view plasmonic microscope, we measured kinetics of peptide ligands binding to different GPCRs (FIG. 8).

Wide-View Plasmonic Imaging of Microarrays

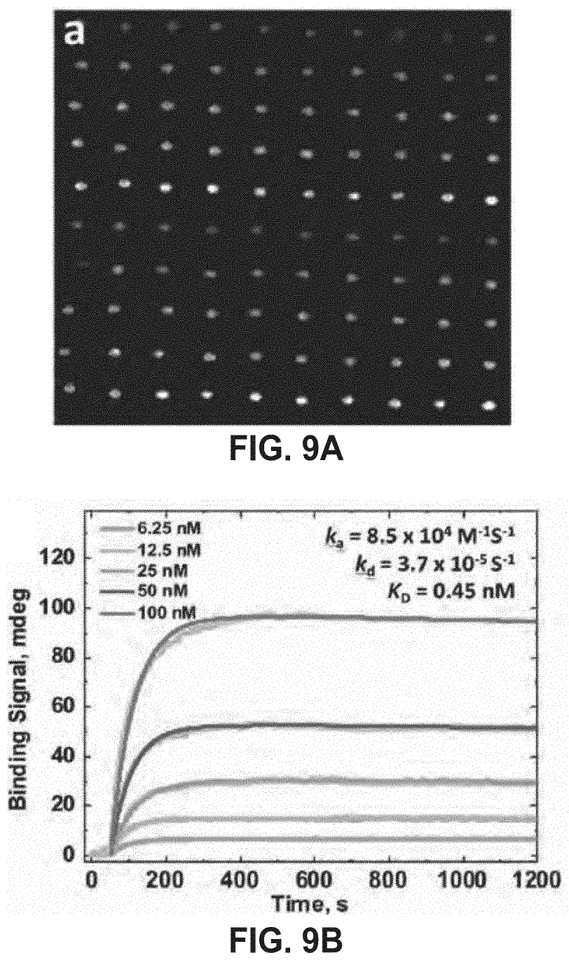

To prepare for high-throughput studies, we have applied the wide-view plasmonic imaging capability to measure protein/protein interactions in microarray format. FIG. 9 shows an image after binding of anti-BSA antibody at 5 different concentrations to a 10×10 array of BSA created with contact free piezo-electrical printing with a spot to spot distance of 800 μm. Protein-protein binding kinetics can be quantified with this system. This wide-view plasmonic imaging technology will be used for high-throughput detection of 315 human GPCRs with the VirD-Oscillators.

The experiments described herein established the working principle of the VirD-Oscillator microarray detection technology and demonstrated its unique capabilities for label-free quantification of both large and small molecule binding to transmembrane proteins. To transform the technology into a high-throughput microarray detection platform, we develop protocols to fabricate a VirD-Oscillator microarray for 315 non-odorant GPCRs, develop and build a wide-view plasmonic detection system to read the microarray, and validate the technology with small molecules, peptides and antibody ligands for cancer relevant research and drug discovery.

VirD-Oscillator Microarray for 315 Non-Odorant Human GPCRs

Produce 315 VirD Non-Odorant Human GPCRs

We developed a GPCR library, including a collection of 315 non-odorant human GPCRs displayed on virions by cloning human GPCRs into the HSV-1 genome using the Gateway method. We focus on these non-odorant GPCRs because unlike odorant/sensory GPCRs they play critical roles in signaling pathways involved in various human diseases, including cancer, and are thus preferred drug targets for treating the diseases. We produce virions displayed with the entire collection of the 315 GPCRs by infected Vero cells following the protocol described herein. We use these virions to test the microarray fabrication protocols and produce VirD-Oscillator chips with these non-odorant human GPCRs for validation.

Develop a VirD-Oscillator Microarray Chip Fabrication Protocol

We develop a reliable protocol to fabricate VirD-Oscillator microarrays. Key features of the protocol include simplicity and reproducibility with >90% yield and batch-to-batch variability <10%.

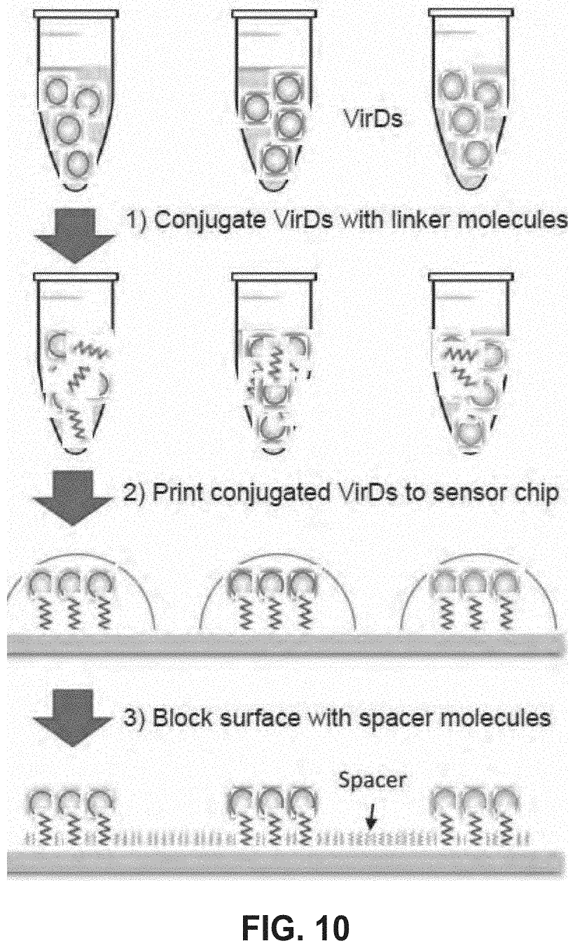

Fabrication of VirD-Oscillator microarray chips: The basic procedure to fabricate a VirD microarray as shown in FIG. 10 includes: 1) Conjugate linker molecules to virions by incubating the virions with HS-PEG-NHS at 1:5 ratio in 1×PBS overnight to form a virion-PEG complex. 2) Print the conjugated virions to the sensor chips using a contact-free microarray spotter and then incubate the chips for 20 min to allow self-assembly of the virion-oscillators on the chip. 3) Rinse the chips with PBS buffer, followed by incubation in a solution containing spacer molecules (e.g., 15 nM HS-PEG6-COOH) overnight to passivate the exposed gold area between virions.

Short and long-term storage of the microarray chips: For short-term storage, the gold chip coated with the virion-oscillators can be kept in buffer and stored at 4° C. However, a long-term storage method will allow use of the technology in other research labs and pharmaceutical companies. We test long-term storage conditions of the chips, starting with the standard protein microarray storage protocols, such as covered with buffer containing glycerol and store in −80° C. freezer. In some embodiments, six months storage without degradation of the performance is reached. We test the chips stored under different conditions to determine an optimal storage strategy.

Determine Key Parameters in the Fabrication of VirD-Oscillator Microarrays

Our experiments indicate that several key conditions, including the type and length of the linker molecules, array spot size and density, and buffer condition, may affect the performance and quality of the VirD-Oscillator microarray. We evaluate these parameters to improve the VirD-Oscillator microarray fabrication for binding kinetics measurements.

Polymer linker type: Each virion is tethered to the gold chip via a flexible polymer linker. Determining an ideal polymer linker is thus important to the performance of the VirD-Oscillator detection technology. We have tested DNA and PEG linkers. DNA is charged, which complicates the interpretation of the data. In some experiments, we focus on PEG, because it is neutral and known to interact weakly with proteins and other biomolecules (reducing non-specific binding). We use commercial bi-functional PEG linkers, such as HS-PEG-NHS, with each opposing end terminated with either a thiol or NHS functional groups. The PEG molecules can thus bind to the gold surface via the thiol-gold bond, leaving the NHS for linking to primary amines on the virions. This strategy was successfully used to immobilize gold nanoparticles on gold chips for other applications. We monitor the VirD-Oscillator assembly process in real time with plasmonic imaging. This helps determine problems during each step, and quantify the coverage of the PEG layer, density of the virions, and yield of the VirD-Oscillator assembly.

Polymer linker length: We fabricate VirD-Oscillator microarrays using PEG with lengths varying from a few tens of nm to a few hundred of nm. A PEG linker with a length of >100 nm will allow the virion to move in and out of the evanescent field completely, thus maximizing the image contrast change of the virion (see FIG. 2b). However, noise from Brownian motion of the virion increases with the linker length, as the virion is able to move over a larger distance. We analyze the dependence of the signal-to-noise ratio on the PEG length and determine the length that provides the best results. PEG with different lengths are readily available from a variety of commercial vendors.

Spacer molecules: We insert spacer molecules in between the PEG linkers to control the VirD-Oscillator density, minimize adhesion of the virions to the chip surface, and reduce non-specific binding. We test thiolate spacer molecules with different lengths and terminal groups, e.g. —COOH, —OH, and —NH₂, and study their impact on the VirD-Oscillator density, surface adhesion, non-specific binding, stability, and shelf life of the array.

Variability of individual VirD-Oscillators: Our experiments revealed that the kinetic and affinity constants, and the maximum binding signal vary from virion to virion, which is likely due to heterogeneity of the displayed GPCRs and variability in the local environment of each GPCR. We examine the origin of the variability by controlling the linker to virion ratio, and the VirD-Oscillator density.

Microarray spot size and density: The optimal spot size and density of the VirD-Oscillator microarray are important practical considerations for achieving the best performance. Our experiments used 6-nL drop volume, which led to a spot diameter of ˜150 μm (FIG. 6). This spot size provided good signal-to-noise ratios for precise binding kinetics measurement. The distance between two adjacent spots in a row or a column was 800 μm. To cover the 315 non-odorant human GPCRs, a 20×20 microarray (with an area of 16×16 mm²) will be sufficient, but the VirD-Oscillator microarray has a plenty of room to expand by optimizing the sport size, density and microarray area.

Buffer: Buffer may change the effective charge of the virions via charge screening. In our tests, we used various buffers (e.g., PBS). In some aspects, we examine the effects of buffer type and ionic strength on the performance of the VirD-Oscillator microarray.

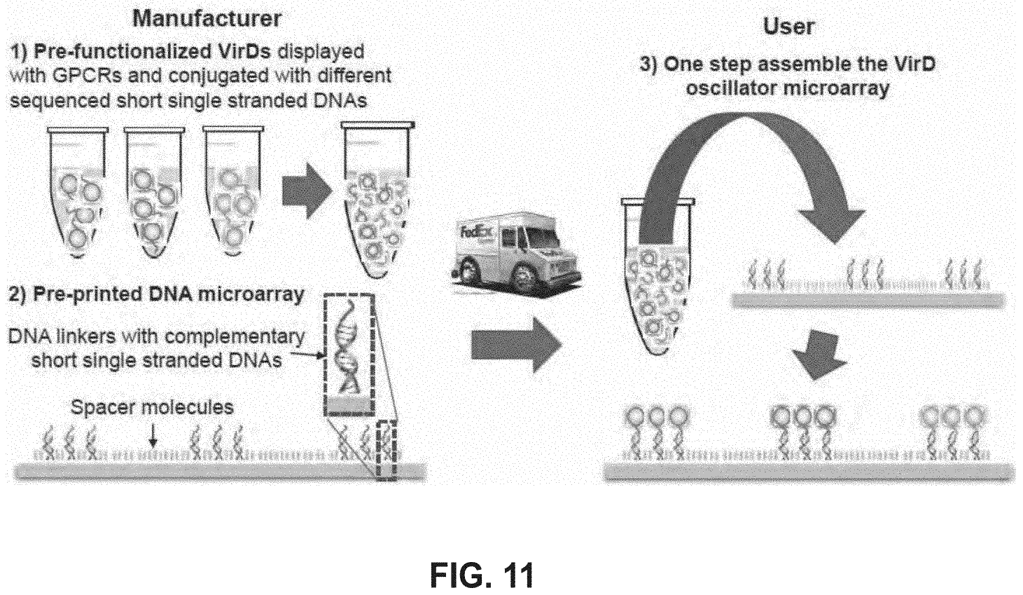

In some embodiments, if long-term storage of pre-printed VirD-Oscillator chips is found problematic, we provide gold chips and virions for the user to print their own microarrays. For users without access to a microarray printer, we develop a DNA-barcoded VirD-Oscillator microarray, where each virion will be attached with a DNA linker to bind to complementary DNA preprinted on the chip. In some embodiments, the fabrication protocol includes the following steps (FIG. 11): (1) Surface-bound DNA linkers with short single-stranded DNA overhangs (10-15 based pair) are pre-printed onto the sensor surface. Fabrication of such pre-printed DNA microarrays is a well-established practice. (2) Virions pre-conjugated with the complementary ssDNA sequences will be provided to the user for target GPCR attachment. (3) The linker conjugated VirD-Oscillators will then be exposed to the pre-spotted DNA array to assemble into a VirD-Oscillator microarray. The annealing temperature for sequence-specific hybridization will be restricted to a range of 30-37° C. via controlling the DNA sequences.

Plasmonic Detection System for Reading VirD Oscillation Microarrays

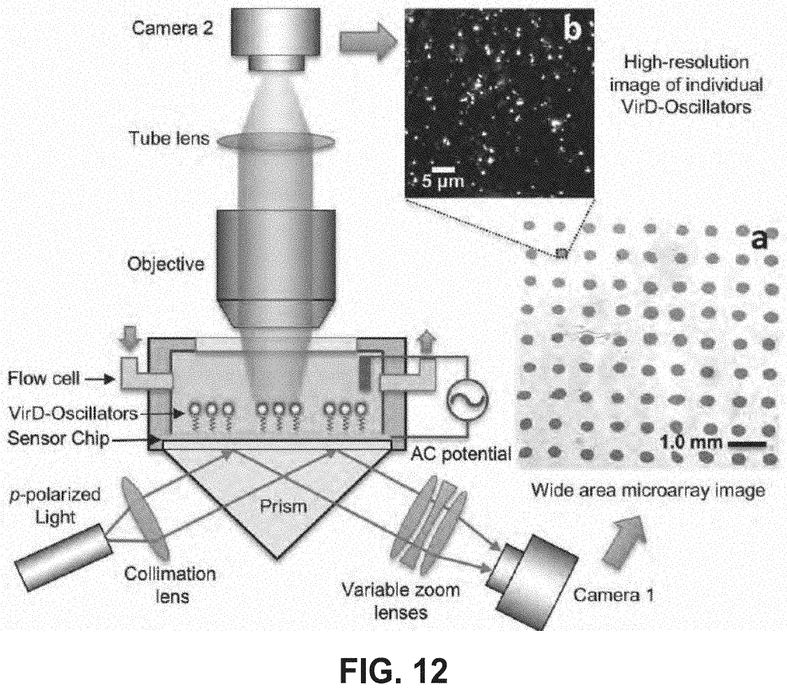

We develop a prototype plasmonic microarray reader for high-throughput quantification of molecular binding kinetics of the membrane proteins displayed on the virions. The microarray reader features a dual imaging capability that can image the entire microarray with a wide-view plasmonic imager from the backside of the microarray chip and resolve each individual VirD-Oscillator from the top of the chip with a high-resolution plasmonic imager (FIG. 12). The former allows simultaneous detection of binding kinetics on each of the spots for high-throughput quantification of ligand binding to the 315 non-odorant human GPCRs in one measurement, but it cannot resolve single virions in each spot. The latter is equipped with a high numerical aperture objective to collect scattered plasmonic signals and image single virions in the microarray. The single-virion detection capability helps study the heterogeneity of different virions and identify possible fabrication flaws in the microarray.

In some embodiments, the dual wide-view and high resolution plasmonic imaging system consists of: 1) optics for plasmonic detection and imaging, electrical circuit to drive the VirD-Oscillators into oscillation, and fluidics for sample delivery; 2) software for data collection and analysis. Key criteria for the design will include performance, manufacturability, and usability.

Optical imaging system: p-polarized light from a solid-state laser is used to excite surface plasmonic waves on the gold chip placed on top of a prism (FIG. 12). The image area will be 40×40 mm to cover the entire VirD-Oscillator array. A fast CMOS imager (Camera 1, e.g. FLIR GS3-U3-23S6M-C with up to 163 fps capture rate at 1920×1200 pixels resolution) will be used to collect light reflected from the backside of the gold chip and scattered by the plasmonic waves. A variable zoom lens placed in front of the CMOS imager will allow adjustment of the imaging area from 4×4 to 40×40 mm² (FIG. 9a). A high numerical aperture (0.42) and high magnification (50×) long (20 mm) working distance objective will be mounted on top of the microarray chip to collect light associated with plasmonic scattering by the single virions and form a high-resolution plasmonic image through a tube lens and a second CMOS imager (Camera 2). The objective has sufficient spatial resolution (<1 μm) to detect signals from individual VirD-Oscillators. Since the object collects the scattered light from the virions only, it provides low background and high contrast images of single virion oscillators (like dark field imaging)(FIG. 9b). With this approach, we have detected and imaged nanoparticles smaller than 60 nm in diameter.

Electrical circuit (driver of the VirD-Oscillators): A custom potential modulation circuit board will be built to drive the VirD-Oscillators into oscillation. The circuit will support direct connectivity of control electrodes with buffered I/O lines to prevent damage from high voltage shock. The circuit will be connected to a data acquisition board.

Temperature control: Temperature stability is important for accurate and reproducible measurement of binding kinetics. We design a temperature control system to support a range of 10 to 40° C. with stability of ±0.01° C. A precision Peltier element is mounted to the prism block for efficient heating and cooling of the sensor chip. A PTC series thermal controller from Wavelength Electronics will be used for setting and controlling of the temperature.

Sample delivery: Reliable binding kinetics measurements also need a low-noise and reproducible sample delivery. This can be achieved with a carefully designed flow cell and a fluidic pump together with an auto-sampler. In some embodiments, the flow cell has integrated electrodes for electrically driving the VirD-Oscillators. A precision syringe pump with in-line degassing will be used to deliver a stable stream of running buffer. In some embodiments, the auto-sampler (ALIAS™ from Spark Holland, Netherlands)) is used for fast and reliable testing of multiple solutions (different ligands and different concentrations).

Data acquisition: In some embodiments, a data acquisition card from National Instruments Inc. (NI) is used to control digital and analog I/O lines.

Data acquisition and analysis software: In some embodiments, data acquisition and analysis software is developed with Matlab and/or c++ for: 1) recording top and bottom plasmonic images along with potential and current; 2) automatically identifying the array spots in the wide-view images, and individual VirD-Oscillators in the high-resolution images; 3) extracting the image intensity of each spot/VirD-Oscillators vs. time; 4) using a Fourier filter to reduce random noise by removing all noise except at the frequency of oscillation, 5) generating binding curves for each spot or virion; and 6) fitting the data with binding kinetics models.

In some aspects, the present disclosure provides an integrated system for high-resolution plasmonic imaging from the top and wide-view imaging from the bottom. The top image has a small view area (a few hundred microns) and captures only a fraction of light from the entire illuminated microarray. In some applications, this may result in insufficient light and large shot noise in the images. If this is found to be an issue, a second light source (super-luminescent diode) introduced to illuminate the view area captured from the top camera (high resolution plasmonic imaging). An important consideration of this solution is local heating of the sample. We have used 100 mW laser to excite surface plasmons and image cells and detected no sign of sample heating effect. Nevertheless, we can monitor possible heating effect by systematically tuning the illumination intensity.

Test and Validate VirD-Oscillator Microarray Technology

In some embodiments, we test and validate VirD-Oscillator microarray technology by measuring the binding kinetics of small molecule drugs, peptide ligands, and antibodies against human GPCRs and validate the measurements with reference technologies when applicable. The success of these tests validate VirD-Oscillator microarray technology for studying transmembrane protein functions (e.g. orphan GPCR functions), discovering new biomarkers and drug targets, and screen new drugs for cancer and other diseases by ranking the drug candidates based on the measured affinity and kinetic constants.

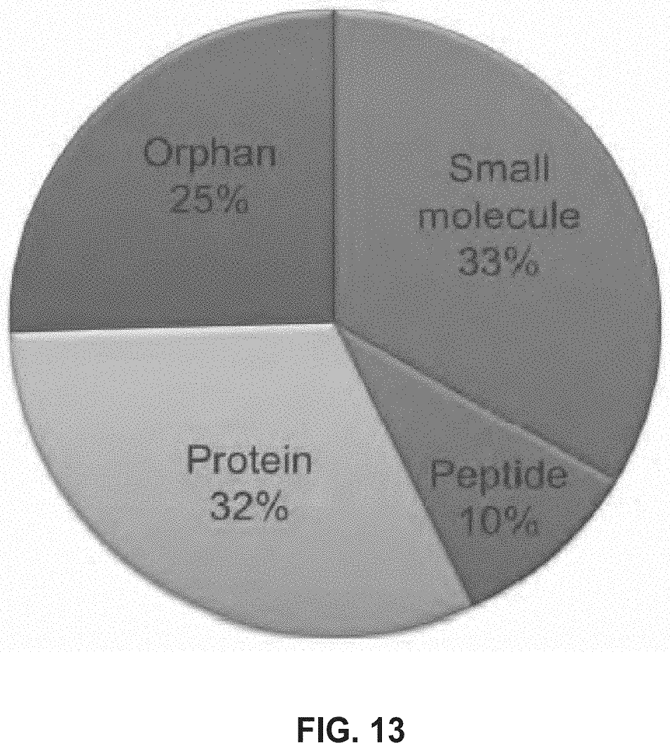

Ligands identified for non-odorant GPCRs consist of a wide variety of molecules, including small molecules, peptides, and proteins (FIG. 13). We test the VirD-Oscillator microarray technology by selecting ligands from each class. This allows us to evaluate the capability of the technology for measuring large and small molecule ligands. We initially focus on 10 GPCRs, including those that are relevant to different types of human cancer. For example, ADRB2, BDKR1, CCRL2 and CXCR2 are well-documented for their roles in cancer growth and/or metastasis, and ACKR3, ADRA2A, CCR7, HRH1, KISS1R, and SSTR1 have been recently identified important for cancer.

Determine Kinetic and Affinity Constants of Ligand Binding to GPCRs

Selection of small molecule and peptide ligands: In some embodiments, to test the usability of the VirD-Oscillator technology we select five small molecule drugs and five peptide ligands with known human GPCR targets (Table 2). Our test of these binding pairs is highly promising. These GPCRs are identified because of their importance in cancer, but their binding kinetic constants are difficult to measure with the existing detection technologies. To evaluate the accuracy, we compare the affinity constants measured by the present VirD-Oscillator technology with those by reference methods. For binding kinetics, we perform various control experiments, and evaluate the precision of our technology via statistical analysis of multiple measurements.

TABLE 2
Selected small molecule and peptide ligands

| GPCR | Ligand | Class |
|---|---|---|
| ADRB2 | Salmeterol | Small molecule |
| HRH1 | Histamine | Small molecule |
| ADRA2A | Atipamezol | Small molecule |
| CHRM3 | Tiotropium | Small molecule |
| OPRM1 | Alvimopan | Small molecule |
| BDKRB1 | Bradykinin | Peptide |
| NTSR1 | Neurotensin | Peptide |
| KISS1R | Kisspeptin | Peptide |
| SSTR1 | Somatostatin | Peptide |
| TACR1 | Substance P | Peptide |

Kinetics measurements: In some embodiments described herein, we first apply an alternating potential to drive the virions into oscillation in PBS buffer at a flow rate of 300 μL/min over the virion-oscillators, and then record the oscillation amplitude to establish a baseline. Once the baseline is stabilized, a ligand will be flowed over the VirD-Oscillator microarray, and the binding of the ligand to the GPCRs on the virion surfaces will be tracked in real-time. Following the binding, unbinding will be studied by flowing PBS buffer. In some embodiments, we repeat this measurement for different concentrations of the ligand. By fitting the binding and unbinding curves obtained at different concentrations with the kinetic model we determine kinetic constants, kₐ and k_{d}, and as well as the affinity, K_{D} (FIGS. 6 and 8). The precision of the VirD-Oscillator microarray measurement will be validated with 3× replicate experiment on separate sensor chips. To confirm specific binding of the ligands with the target GPCRs, we perform negative control experiments using K082, a gB null HSV-1 virion with no GPCR displayed on the envelope. We also perform positive control experiments using D1 antagonist, whose affinity to its canonical GPCR DRD1 was already determined (FIG. 6a).

Results and data interpretation: In some embodiments, we obtain the binding kinetic constants (kₐ and k_{d}) and affinity constant (K_{D}) of the 10 ligands against their canonical GPCRs (Table 2). We compare the binding affinity with the values in the binding database. Kinetic data are difficult to obtain with any of the established methods, we thus focus on precision evaluation by performing statistical analysis on the data obtained by repeating the measurement with six different chips for each binding pair.

An additional consideration is that a small molecule ligand may bind to more than one GPCR. However, we expect that the kinetic and affinity constants will be different. Surveying the binding of a small molecule with many non-odorant GPCRs in a single assay allows us to rank the binding affinity and kinetics of the small molecules with different GPCRs, which is important for drug screening.

Measure Kinetic and Affinity Constants of Antibody Binding to GPCRs

Selection of antibodies: Antibody drugs are often designed based on the structures of the canonical ligands, and they may suffer from unwanted side effects associated with off-target binding activities. This is mainly because some GPCRs share high sequence homology in the binding pockets targeted by the drug. Therefore, recent efforts have been devoted to developing antibody-based biologicals targeting ectodomains of the non-odorant GPCRs. The capability of measuring binding kinetics and specificity of anti-GPCR antibodies simultaneously with the VirD-Oscillator array platform will be able to identify these issues early in the development process, thus reducing overall drug development time and cost.

In some embodiments, we select five commercially available mAbs (Table 3) on the basis of preliminary studies using the GPCR VirD arrays (see FIG. 4). Four of them targeting CXCR1/2/5 and CCR7 show ultra-specific binding affinity, while anti-ACKR3 has two off-targets (i.e., CALCR and GPR61) in addition to its intended target. Both cell surface staining (immunofluorescence assays) and immunoblot analysis confirmed the weaker off-target interactions (FIG. 14). It is insightful to compare the differences in binding kinetics between the canonical and off-targets of this mAb. Additional reasons for choosing these mAbs includes 1) The targeting GPCRs recognize chemokines as their canonical ligands; 2) Some of the GPCRs (e.g., CXCR1 and CXCR2) recognize the same ligand (e.g., IL8); and 3) CXCR1, CXCR2, and CXCR5 share a significant amount of sequence homology. Quantitative binding kinetic study helps to understand the structure and function of these GPCRs.

TABLE 3
Select

[symbol]

| mAb targets | Catalog No. | Function data |
|---|---|---|
| CCR7 | R&D MAB197 | EC50 = 5 μg/mL |
| CXCR1 | R&D MAB330 | EC50 = 2 μg/mL |
| CXCR2 | R&D MAB331 | EC50 = 5 μg/mL |
| CXCR5 | R&D MAB190 | EC50 = 1.5 μg/mL |
| ACKR3 | R&D MAB4139 | Not available |

[symbol]

indicates data missing or illegible when filed

[symbol] and affinity [symbol] An important [symbol] each of the [symbol] concentration [symbol] for the [symbol] some embodiments, we also carry out negative and positive control experiments to validate the results. We typically compare VirD-Oscillator microarray measured binding affinity results with the values obtained from cell-based neutralization test to validate our technology.

Results and data interpretation: In some embodiments, we obtain both the kinetic constants and affinity values for the mAbs. We expect that the binding affinity of anti-ACKR3 to the two off-target GPCRs, namely CALCR and GPR61, to be weaker than that of ACKR3. In some embodiments, we also compare the kinetic constants for the on- and off-target GPCRs and examine the correlation between the kinetic constants and affinity data. Additionally, we determine the precision by repeated measurements on six different chips for each binding pair listed in Table 3.

In some aspects, the present disclosure provides robust VirD-Oscillator chip fabrication protocol developed with >90% yield, <10% batch-to-batch variation and >6 months shelf life. In some embodiments, the optical detection system is built to measure binding kinetics of 315 virions displayed non-odorant human GPCRs on a single chip, with a ligand detection limit <500 Da, and response time of 1 second. In some embodiments, binding kinetics of selected 5 small molecules, 5 peptides and 5 antibodies targeting GPCRs related to tumorigenesis and metastasis are measured with precision quantified and off-target binding events determined.

Plasmonic Scattering Microscopy Setup for Single Oscillator Detection

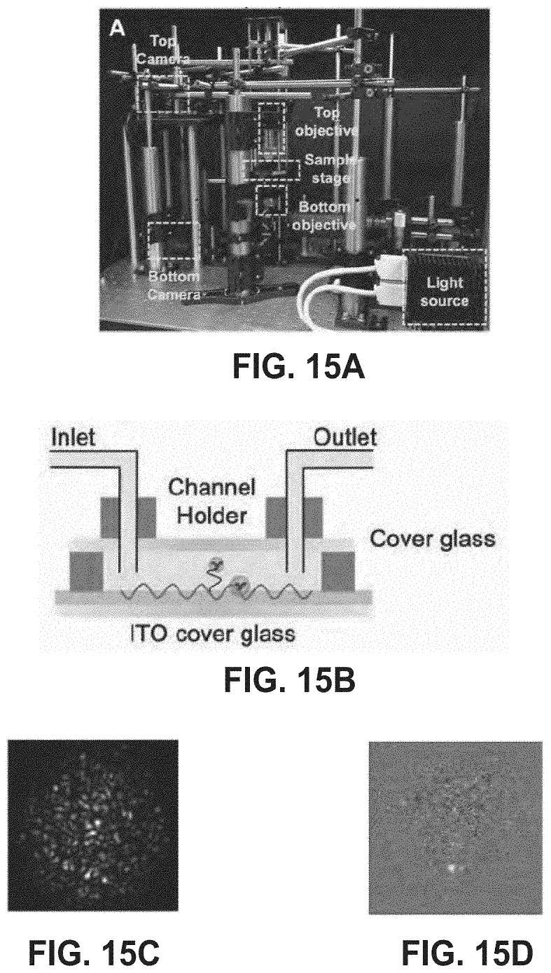

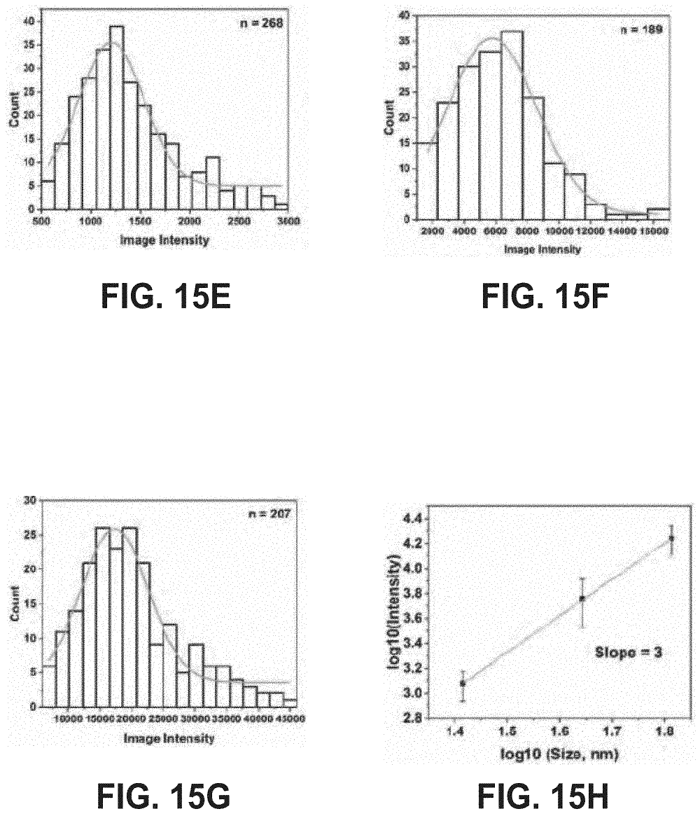

In some embodiments, a plasmonic scattering microscopy (PSM) with single molecule detection ability has been implemented for detection of single virion oscillator. The setup (FIG. 15A) includes two objectives on the bottom and top of the sample stage, and a flow cell (FIG. 15B) is used for loading the sample. Light is directed from the bottom and reach the total internal reflection on the sample slide to excite surface plasmonic wave; at the same time, focus the top objective on the surface of sample slide, a plasmonic scattering image (FIG. 15C) can be observed. Due to the low background illumination and interference between the background scattered light and the object scattered light, the sensitivity of plasmonic scattering imaging can achieve the detection of single proteins (1). The plasmonic scattering image also has improved spatial resolution over SPR image for higher density detection of oscillators. Inject sample through flow cell and monitor the change through top objective, 26 nm, 44 nm and 65 nm polystyrene nanoparticles were measured to establish a calibration curve. Subtract previous frame in original video to get the differential image (FIG. 15D) and count the intensity of nanoparticles in every frame to get the statistics results (FIG. 15E to G) Then fit the statistics results by Gaussian distribution and use center intensity to get the calibration result. As shown in FIG. 15H, the image intensity change caused by a particle fall on the surface is proportional to the cubic power of its size, indicating the interference signal is dominant.

Producing Virion Displayed GPCRs for VirD-Oscillator Assembly

A couple of methods to tag single-stranded DNA oligos to purified virions have been tested. On the basis of Cyber Gold stain, the method that utilizes a crosslinker MSCC looked promising. The reaction conditions can be further optimized for better labeling efficiency.

Demonstrated Single Oscillator Detection by Plasmonic Scattering Microscopy

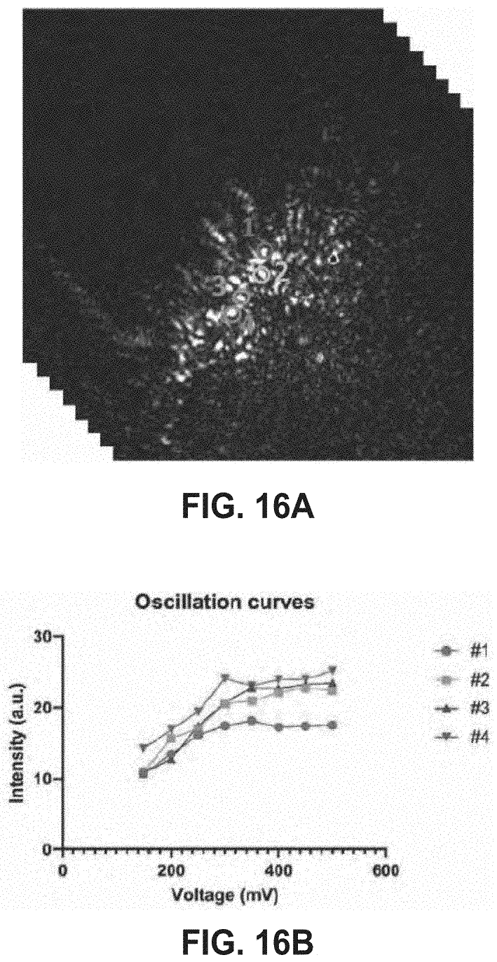

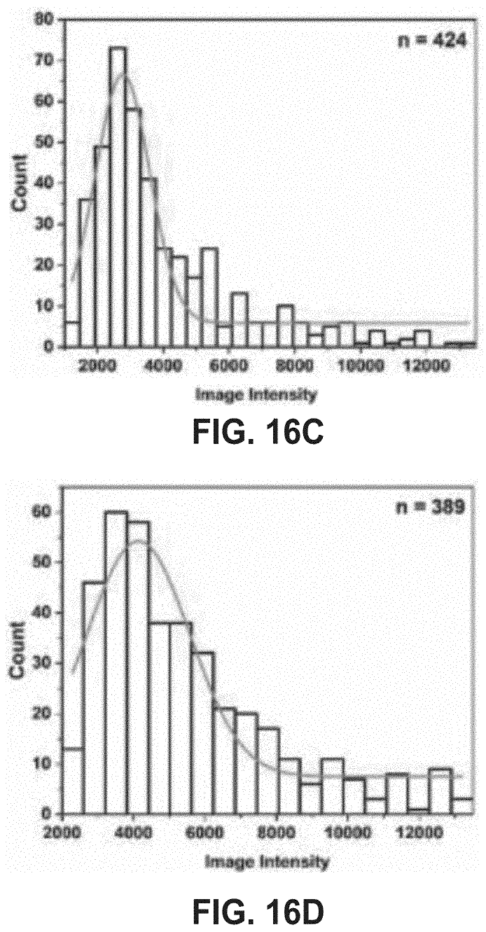

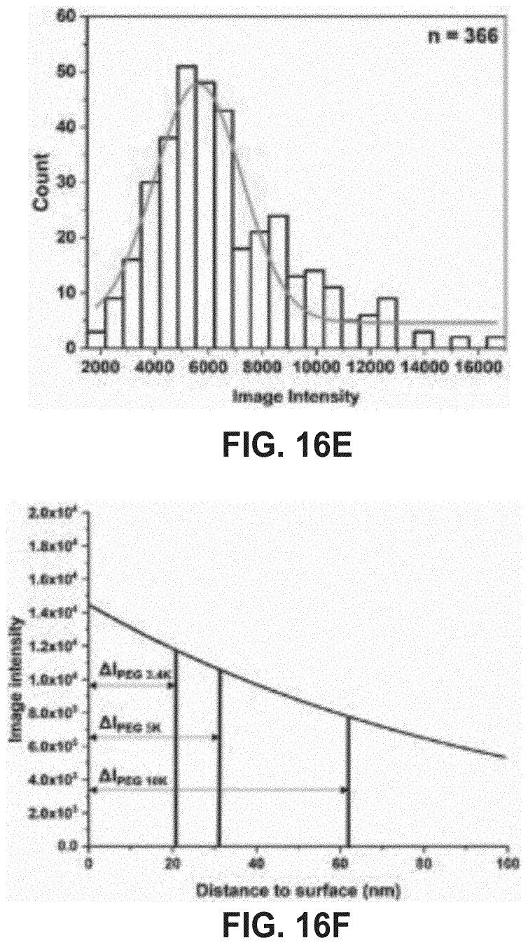

We tested the plasmonic scattering imaging of single oscillator with a large IgM protein to mimic the virion. PEG with molecular weight 10K was used as the molecular linker, one end is functionalized by NHS, which can bind with protein and the other end can choose different groups to connect with substrate. A 10:1 protein and linker molecule ratio was used to ensure most of the oscillator just has one linker. Use a three-electrode system to apply an alternative electric field to drive the oscillator and take the video with a frame rate meets sampling theorem. FIGS. 16A & B show the response of oscillator at driving frequency and the amplitude change at different electric field intensities. When the electric field is weak, the tethered molecule moves within the range of linker length, a linear region appears; when the electric field gets stronger, the movement of oscillator is limited by the linker, so a plateau appears. To ensure the electric force is sufficient and the linker works as expected, we also tested linkers with different length. FIG. 16C to E show the statistics of plateau intensity with linker PEG 3.4K, 5K and 10K. The intensity changes fit well with the decay of evanescent field (FIG. 16F), it indicates that the linker can be fully stretched and confine the movement of oscillator. The long linker PEG 10K provides a larger signal to noise ratio and good linear response and can be used in oscillator assembly protocols.

Developed an Oscillator Storage Method

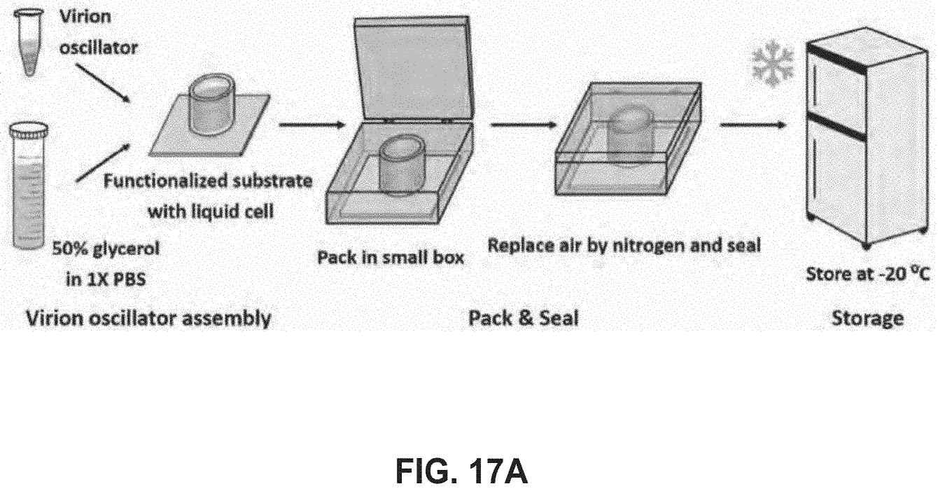

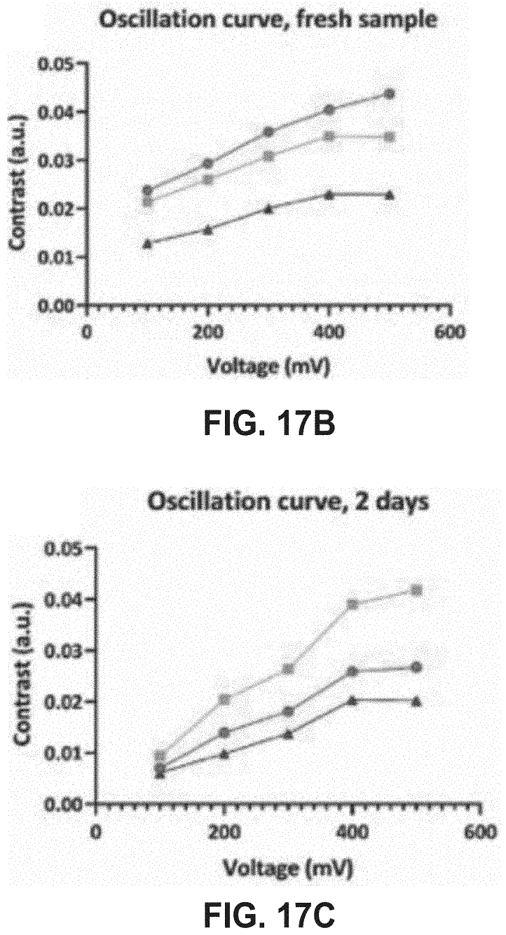

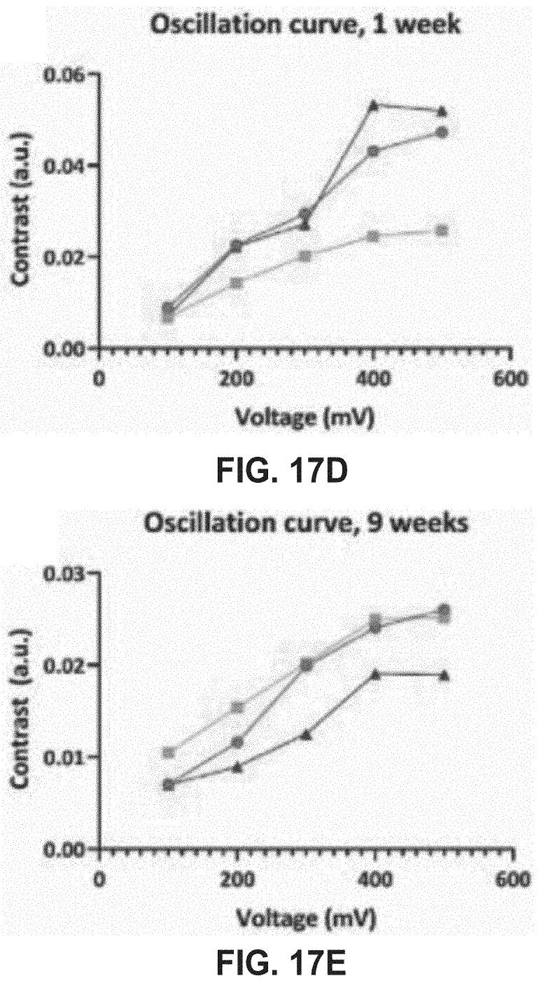

Proper shelf life of pre-functionalized VirD-oscillator is key important for commercialization of the VirD Oscillator technology. Based on common storage methods for virion and chemicals, we tested the workflow shown in FIG. 17A for storage of prefabricated oscillator chips. Essentially, we use 50% glycerol to protect the protein molecules and keep the buffer in liquid state for freezing storage. Nitrogen was also filled in to reduce the oxidation rate. Three storage periods have been tested (FIG. 17C to 17E) and compared with the fresh oscillator result (FIG. 17B). Single oscillator can be observed in each sample, illustrating that the storage condition is capable to reduce the degradation and extend the shelf life of oscillator samples. A six-month and longer storage periods are optionally utilized in some embodiments.

Large View Area Plasmonic Imaging System and a Flow Cell with Electrical Field Modulation Capability for Detection of Viral Oscillator Microarray

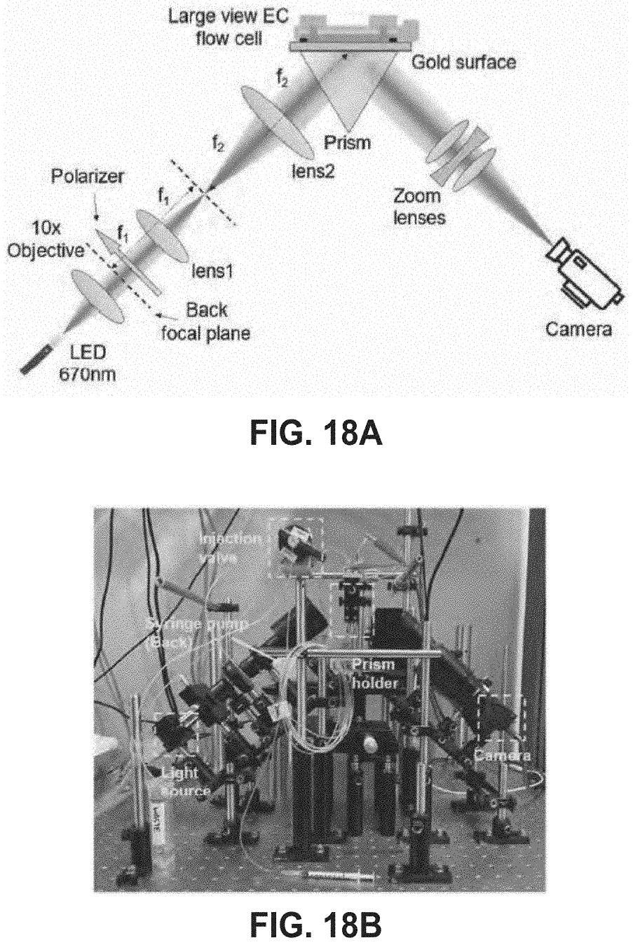

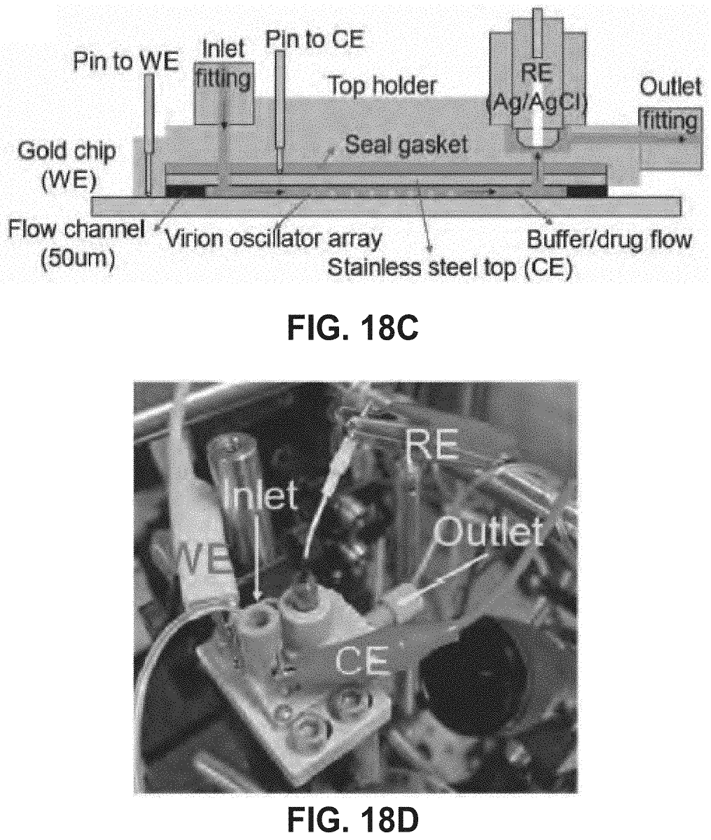

A prism-based large view area plasmonic imaging system has been implemented for virion oscillator microarray detection. The system (FIG. 18) is based on the Kretschmann configuration. A maximum of 15 mm by 15 mm detection area can be achieved in this configuration that sufficient to measure up to 400 spots of oscillator microarray. Because the working area is much larger than traditional ensemble detection, an evenly sample delivery is critical for the kinetics measurements. We designed a flow cell with electrical field modulation capability for this intention, as shown in FIGS. 18C and 18D. A laser cut double side tape forms the flow channel between the gold sensor chip and counter electrode top cover. The sensor chip is pressed on the prism with a top holder by screws. Running buffer is injected by a syringe pump through an injection valve, which has a separate sample loop for smooth sample delivery. A brief workflow of the system is as the following: 1) immobilize the virion oscillator on gold surface; 2) assemble the flow cell on the prism; 3) record the plasmonic images with potential modulation; 4) extract the spot intensity variations over time from the image stack; 5) extract oscillation amplitude with fast Fourier transform; 6) Fit the oscillation amplitude curves to obtain the binding kinetics.

Implemented the Surface Chemistry for Virion Oscillator Fabrication

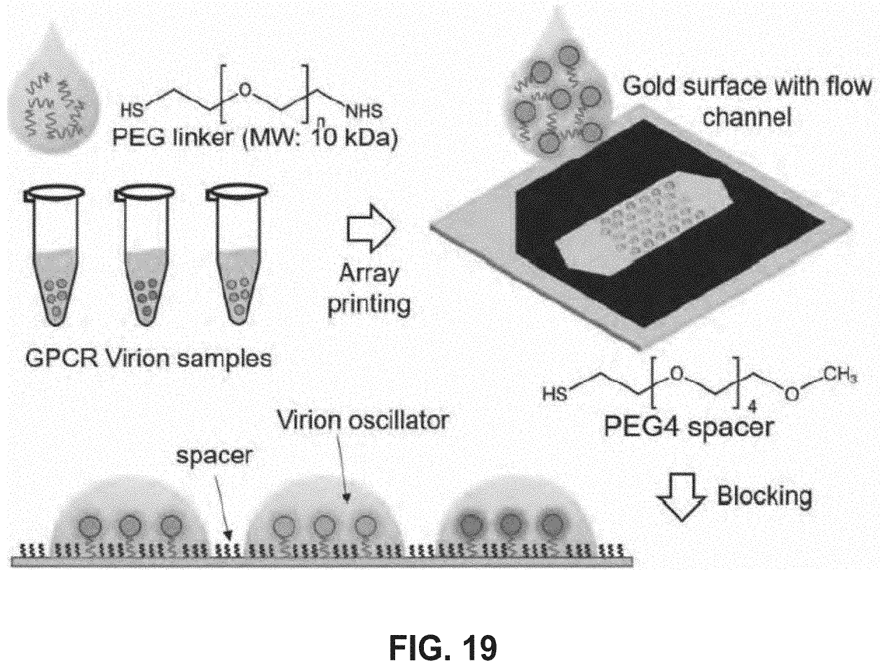

Immobilize the virion oscillator on gold is the key to fabricate the sensor chip. Ideally, the linker is flexible and contribute little mass and charge to the oscillator than the virion. Based on these conditions, we chose the polyethylene glycol (PEG) as the linker (MW: 10 kDa). To bind with both gold and virion, the thiol group and N-Hydroxysuccinimide (NHS) compound are modified on the two ends of the PEG molecule. Thiol can form a strong bond with gold and NHS can react with the amine groups in virion's shell protein. FIG. 19 shows the surface chemistry workflow for fabricating the virion oscillators. Briefly: 1) Add the PEG solution into the virion stocking buffer at a molecule number ratio about 10 to 1 and incubate the mixture overnight at 4° C. to conjugate the linkers to virions. 2) Spotting the virion with linker to a clean bare gold surface (47 nm gold on glass coverslip, cleaned by ethanol and DI water and annealed by hydrogen flame) for a half hour incubation. 3) Apply a shorter PEG molecule with thiol to block the rest of gold surface to minimize non-specific binding.

Tested Selected GPCRs Oscillators on the Large View Plasmonic Imaging System

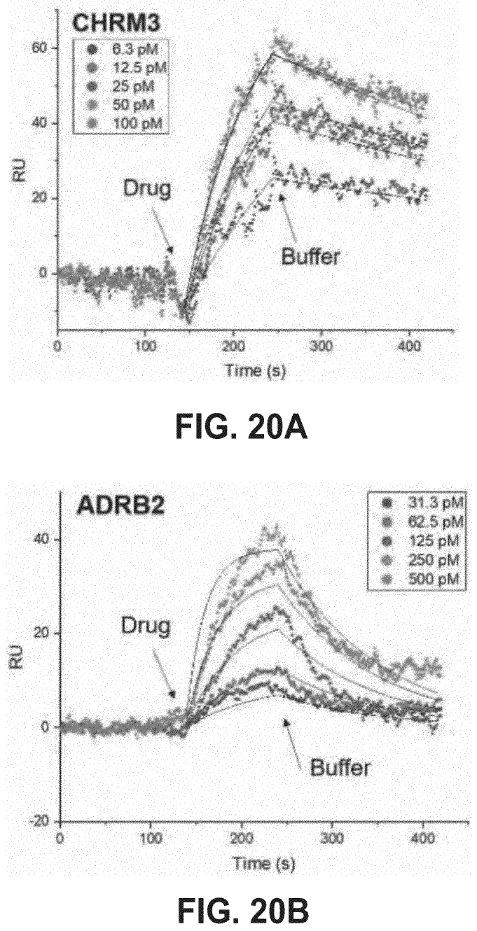

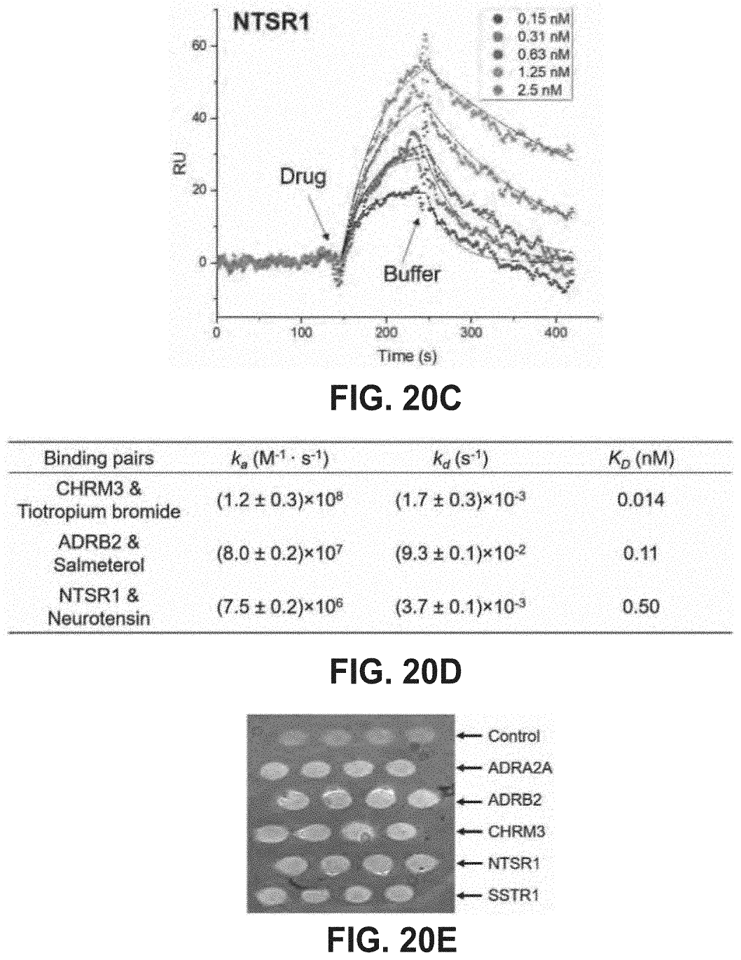

To test the prototype plasmonic imaging system and virion oscillator fabrication protocol, we measured selected GPCRs oscillators in a hand spotted virion oscillator array format. FIGS. 20A to 20C show the oscillation amplitude of 3 kinds of GPCR virion (CHRM3, ADRB2 and NTSR1) to their corresponding ligands (tiotropium bromide, salmeterol and neurotensin). The colored dots are the virion oscillation amplitude calculated from Fourier transform at each second and the solid line is the exponential fitting of binding kinetics. The fitting results are summarized in the FIG. 20D. We also tested printed oscillator microarray using a contact free microarray printer (FIG. 20E).

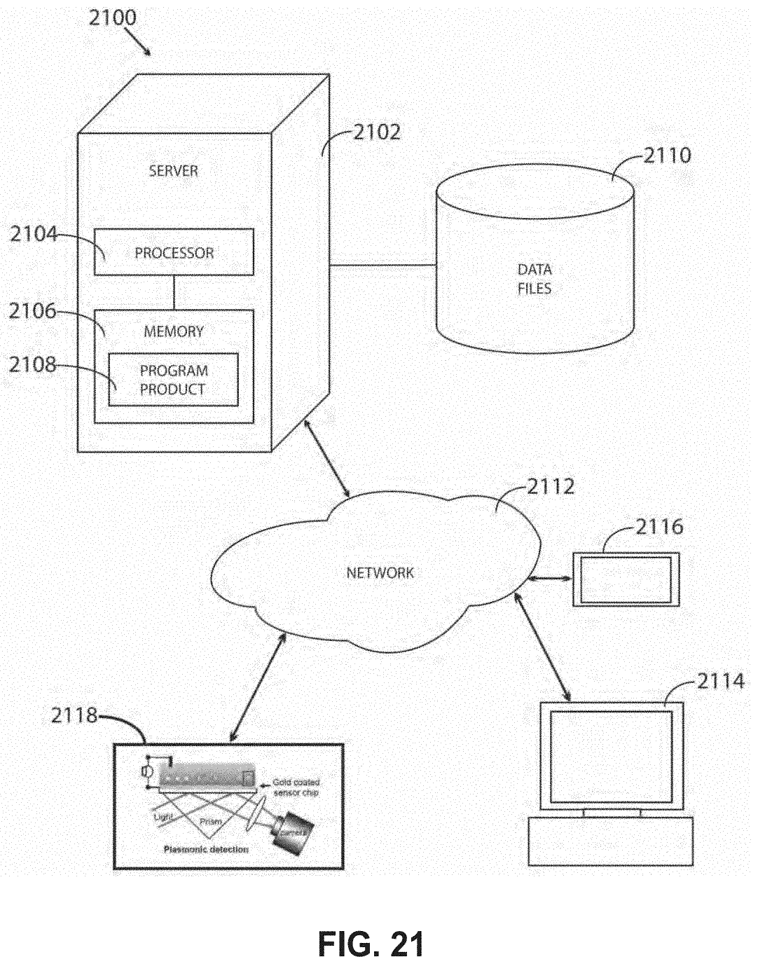

The present disclosure also provides various systems and computer program products or machine readable media. In some aspects, for example, the methods described herein are optionally performed or facilitated at least in part using systems, distributed computing hardware and applications (e.g., cloud computing services), electronic communication networks, communication interfaces, computer program products, machine readable media, electronic storage media, software (e.g., machine-executable code or logic instructions) and/or the like. To illustrate, FIG. 21 provides a schematic diagram of an exemplary system suitable for use with implementing at least aspects of the methods disclosed in this application. As shown, system 2100 includes at least one controller or computer, e.g., server 2102 (e.g., a search engine server), which includes processor 2104 and memory, storage device, or memory component 2106, and one or more other communication devices 2114, 2116, (e.g., client-side computer terminals, telephones, tablets, laptops, other mobile devices, etc. (e.g., for receiving molecular interaction data sets or results, etc.) in communication with the remote server 2102, through electronic communication network 2112, such as the Internet or other internetwork. Communication devices 2114, 2116 typically include an electronic display (e.g., an internet enabled computer or the like) in communication with, e.g., server 2102 computer over network 2112 in which the electronic display comprises a user interface (e.g., a graphical user interface (GUI), a web-based user interface, and/or the like) for displaying results upon implementing the methods described herein. In certain aspects, communication networks also encompass the physical transfer of data from one location to another, for example, using a hard drive, thumb drive, or other data storage mechanism. System 2100 also includes program product 2108 (e.g., for determining binding kinetics of a ligand as described herein) stored on a computer or machine readable medium, such as, for example, one or more of various types of memory, such as memory 2106 of server 2102, that is readable by the server 2102, to facilitate, for example, a guided search application or other executable by one or more other communication devices, such as 2114 (schematically shown as a desktop or personal computer). In some aspects, system 2100 optionally also includes at least one database server, such as, for example, server 2110 associated with an online website having data stored thereon (e.g., entries corresponding to molecular interaction data, etc.) searchable either directly or through search engine server 2102. System 2100 optionally also includes one or more other servers positioned remotely from server 2102, each of which are optionally associated with one or more database servers 2110 located remotely or located local to each of the other servers. The other servers can beneficially provide service to geographically remote users and enhance geographically distributed operations.

As understood by those of ordinary skill in the art, memory 2106 of the server 2102 optionally includes volatile and/or nonvolatile memory including, for example, RAM, ROM, and magnetic or optical disks, among others. It is also understood by those of ordinary skill in the art that although illustrated as a single server, the illustrated configuration of server 2102 is given only by way of example and that other types of servers or computers configured according to various other methodologies or architectures can also be used. Server 2102 shown schematically in FIG. 21, represents a server or server cluster or server farm and is not limited to any individual physical server. The server site may be deployed as a server farm or server cluster managed by a server hosting provider. The number of servers and their architecture and configuration may be increased based on usage, demand and capacity requirements for the system 2100. As also understood by those of ordinary skill in the art, other user communication devices 2114, 2116 in these aspects, for example, can be a laptop, desktop, tablet, personal digital assistant (PDA), cell phone, server, or other types of computers. As known and understood by those of ordinary skill in the art, network 2112 can include an internet, intranet, a telecommunication network, an extranet, or world wide web of a plurality of computers/servers in communication with one or more other computers through a communication network, and/or portions of a local or other area network.

As further understood by those of ordinary skill in the art, exemplary program product or machine readable medium 2108 is optionally in the form of microcode, programs, cloud computing format, routines, and/or symbolic languages that provide one or more sets of ordered operations that control the functioning of the hardware and direct its operation. Program product 2108, according to an exemplary aspect, also need not reside in its entirety in volatile memory, but can be selectively loaded, as necessary, according to various methodologies as known and understood by those of ordinary skill in the art.

As further understood by those of ordinary skill in the art, the term “computer-readable medium” or “machine-readable medium” refers to any medium that participates in providing instructions to a processor for execution. To illustrate, the term “computer-readable medium” or “machine-readable medium” encompasses distribution media, cloud computing formats, intermediate storage media, execution memory of a computer, and any other medium or device capable of storing program product 2108 implementing the functionality or processes of various aspects of the present disclosure, for example, for reading by a computer. A “computer-readable medium” or “machine-readable medium” may take many forms, including but not limited to, non-volatile media, volatile media, and transmission media. Non-volatile media includes, for example, optical or magnetic disks. Volatile media includes dynamic memory, such as the main memory of a given system. Transmission media includes coaxial cables, copper wire and fiber optics, including the wires that comprise a bus. Transmission media can also take the form of acoustic or light waves, such as those generated during radio wave and infrared data communications, among others. Exemplary forms of computer-readable media include a floppy disk, a flexible disk, hard disk, magnetic tape, a flash drive, or any other magnetic medium, a CD-ROM, any other optical medium, punch cards, paper tape, any other physical medium with patterns of holes, a RAM, a PROM, and EPROM, a FLASH-EPROM, any other memory chip or cartridge, a carrier wave, or any other medium from which a computer can read.

Program product 2108 is optionally copied from the computer-readable medium to a hard disk or a similar intermediate storage medium. When program product 2108, or portions thereof, are to be run, it is optionally loaded from their distribution medium, their intermediate storage medium, or the like into the execution memory of one or more computers, configuring the computer(s) to act in accordance with the functionality or method of various aspects disclosed herein. All such operations are well known to those of ordinary skill in the art of, for example, computer systems.

In some aspects, program product 2108 includes non-transitory computer-executable instructions which, when executed by electronic processor 2104, perform at least: applying an alternating current electric field to a substrate having a first surface and a second surface opposite the first surface, wherein the first surface comprises an electrically conductive coating and a population of virions connected to the first surface via one or more linker moieties, wherein viral envelopes of the virions display one or more receptors that bind, or are capable of binding, to a ligand, which alternating current electric field induces the virions to oscillate proximal to the first surface of the substrate using the power source; introducing an incident light toward the second surface of the substrate from the light source to induce the plasmonic wave at least proximal to the first surface of the substrate; and detecting changes in oscillation amplitudes of the virions over a duration to thereby determine the binding kinetics of the ligand.

In some embodiments, binding kinetics of a ligand is measured using device 2118. As shown, device 2118 includes a substrate (e.g., gold coated coverglass) having first surface and a second surface opposite the first surface. The first surface comprises an electrically conductive coating (e.g., Au) and a population of virions connected to the first surface via one or more linker moieties in which viral envelopes of the virions display one or more receptors (e.g., GPCRs or the like) that bind, or are capable of binding, to the ligand. Device 2118 also includes a power source electrically connected to the substrate. The power source is configured to apply an alternating current electric field to the substrate. Device 2118 also includes a prism disposed proximal to the second surface of the substrate. In addition, device 2118 also includes a light source configured to introduce light (e.g., collimated light) through the prism to induce a plasmonic wave at least proximal to the first surface of the substrate, and a detector (e.g., a CMOS camera) configured to collect light reflected from the substrate.

Although this disclosure contains many specific embodiment details, these should not be construed as limitations on the scope of the subject matter or on the scope of what may be claimed, but rather as descriptions of features that may be specific to particular embodiments. Certain features that are described in this disclosure in the context of separate embodiments can also be implemented, in combination, in a single embodiment. Conversely, various features that are described in the context of a single embodiment can also be implemented in multiple embodiments, separately, or in any suitable sub-combination. Moreover, although previously described features may be described as acting in certain combinations and even initially claimed as such, one or more features from a claimed combination can, in some cases, be excised from the combination, and the claimed combination may be directed to a sub-combination or variation of a sub-combination.

Particular embodiments of the subject matter have been described. Other embodiments, alterations, and permutations of the described embodiments are within the scope of the following claims as will be apparent to those skilled in the art. While operations are depicted in the drawings or claims in a particular order, this should not be understood as requiring that such operations be performed in the particular order shown or in sequential order, or that all illustrated operations be performed (some operations may be considered optional), to achieve desirable results.

Accordingly, the previously described example embodiments do not define or constrain this disclosure. Other changes, substitutions, and alterations are also possible without departing from the spirit and scope of this disclosure.

What is claimed is:

1. A method of determining binding kinetics of a ligand, the method comprising:
contacting the ligand with a first surface of a substrate, which first surface comprises an electrically conductive coating and a population of virions connected to the first surface via one or more linker moieties, wherein viral envelopes of the virions display one or more proteins that bind, or are capable of binding, to the ligand;
applying an alternating current electric field to the substrate to induce the virions to oscillate proximal to the first surface of the substrate; and,
detecting changes in oscillation amplitudes of the virions over a duration, thereby determining the binding kinetics of the ligand.

2. The method of claim 1, comprising detecting the changes in the oscillation amplitudes of the virions using a plasmonic imaging technique and/or a microscopic imaging technique.

3. The method of claim 1, wherein:
the electrically conductive coating comprises gold (Au), indium tin oxide (ITO), silver (Ag), copper (Cu), and/or aluminum (Al); and/or,
the linker moieties comprise polyethylene glycol (PEG) moieties and/or biomolecules.

4. The method of claim 1, wherein:
the virions comprise human herpes simplex virus-1 (HSV-1) virions; and/or,
the proteins comprise a G-protein-coupled receptor (GPCR).

5. The method of claim 1, further comprising transfecting the virions with nucleic acid vectors that encode the proteins prior to the contacting step.

6. The method of claim 1, comprising quantifying the binding kinetics and binding affinity of the ligand using the detected changes in the oscillation amplitudes of the virions over the duration.

7. The method of claim 1, comprising determining the binding kinetics of the ligand in substantially real-time.

8. The method of claim 1, wherein the detecting step comprises introducing an incident light toward a second surface of the substrate to induce a plasmonic wave at least proximal to the first surface of the substrate and detecting a change in intensity of the incident light reflected at an interface of the first surface of the substrate.

9. The method of claim 1, comprising introducing the incident light via at least one objective lens and/or at least one prism.

10. The method of claim 1, comprising introducing the incident light using a superluminescent diode (SLED).

11. The method of claim 1, comprising detecting the changes in the oscillation amplitudes of the virions over the duration using a CMOS camera.

12. A virion oscillator array device, comprising a substrate that comprises a first surface that comprises an electrically conductive coating and a population of virions connected to the first surface via one or more linker moieties, wherein viral envelopes of the virions display one or more proteins that bind, or are capable of binding, to a ligand.

13. The virion oscillator array device of claim 12, wherein:
the electrically conductive coating comprises gold (Au), indium tin oxide (ITO), silver (Ag), copper (Cu), and/or aluminum (Al); and/or,
the linker moieties comprise polyethylene glycol (PEG) moieties and/or biomolecules.

14. The virion oscillator array device of claim 12, further comprising one or more spacer moieties connected to the first surface and/or to the linker moieties.

15. The virion oscillator array device of claim 12, wherein:
the virions comprise human herpes simplex virus-1 (HSV-1) virions; and/or,
the proteins comprise a G-protein-coupled receptor (GPCR).

16. A system for determining binding kinetics of a ligand, comprising:
a substrate having a first surface and a second surface opposite the first surface, wherein the first surface comprises an electrically conductive coating and a population of virions connected to the first surface via one or more linker moieties, wherein viral envelopes of the virions display one or more proteins that bind, or are capable of binding, to the ligand;
a power source electrically connected to the substrate, which power source is configured to apply an alternating current electric field to the substrate;
an objective lens or a prism disposed proximal to the second surface of the substrate;
a light source configured to introduce light through the objective lens or the prism to induce a plasmonic wave at least proximal to the first surface of the substrate;
a detector configured to collect light reflected from the substrate; and
a controller that comprises, or is capable of accessing, computer readable media comprising non-transitory computer-executable instructions which, when executed by at least one electronic processor, perform at least:
applying an alternating current electric field to the substrate to induce the virions to oscillate proximal to the first surface of the substrate using the power source;
introducing an incident light toward the second surface of the substrate from the light source to induce the plasmonic wave at least proximal to the first surface of the substrate; and,
detecting changes in oscillation amplitudes of the virions over a duration to thereby determine the binding kinetics of the ligand.

17. The system of claim 16, wherein:
the electrically conductive coating comprises gold (Au), indium tin oxide (ITO), silver (Ag), copper (Cu), and/or aluminum (Al)
the linker moieties comprise polyethylene glycol (PEG) moieties and/or biomolecules.

18. The system of claim 16, further comprising one or more spacer moieties connected to the first surface and/or to the linker moieties.

19. The system of claim 16, wherein:
the virions comprise human herpes simplex virus-1 (HSV-1) virions; and/or,
the proteins comprise a G-protein-coupled receptor (GPCR).

20. The system of claim 16, wherein the non-transitory computer-executable instructions which, when executed by the electronic processor, further perform at least: quantifying the binding kinetics and binding affinity of the ligand using the detected changes in the oscillation amplitudes of the virions over the duration.
